(19) United States
(12) Patent Application Publication
(10) Pub. No.: US 2026/0102351 A1
(43) Pub. Date: Apr. 16, 2026

(54) LYOPHILIZED FORMULATION OF CROFELEMER AND METHODS OF TREATMENT USING THE SAME

(71) Applicant: NAPO PHARMACEUTICALS, INC., San Francisco, CA (US)

(72) Inventors: Pravin Chaturvedi, Andover, MA (US); Mark Johnson, Fayetteville, NC (US); David Sesin, Eagle, ID (US)

(21) Appl. No.: 19/116,891

(22) PCT Filed: Sep. 28, 2023
(86) PCT No.: PCT/US2023/034050

§ 371 Date: Mar. 28, 2025

Related U.S. Application Data
(60) Provisional application No. 63/410,988, filed on Sep. 28, 2022.

(51) Int. Cl. A61K 9/19; A61K 31/353; A61K 36/47; A61K 47/12; A61K 47/26; A61P 1/00; A61P 1/12

(52) U.S. Cl. CPC A61K 9/19; A61K 31/353; A61K 36/47; A61K 47/12; A61K 47/26; A61P 1/00; A61P 1/12

(57) ABSTRACT

Presented herein are formulations comprising lyophilized crofelemer, methods of treating short bowel syndrome (SBS), diarrhea associated with SBS, congenital diarrhea disorders (CDDs), or diarrhea associated with CDDs, such as Microvillus Inclusion Disease (MVID) and Tufting Enteropathy, and methods of making formulations comprising lyophilized crofelemer.

### FIELD OF THE INVENTION

The present invention is directed to formulations comprising lyophilized crofelemer, methods of treating short bowel syndrome (SBS), diarrhea associated with SBS, congenital diarrhea disorders (CDDs), namely, Microvillus Inclusion Disease (MVID) and Tufting Enteropathy (TE), or diarrhea associated therewith and methods of making formulations comprising lyophilized crofelemer.

### BACKGROUND

### Short Bowel Syndrome

Short bowel syndrome (SBS) is a complex condition characterized by malabsorption of fluids and nutrients due to congenital deficiencies, premature birth or surgical resection of small bowel segments. Consequently, patients suffer from symptoms such as diarrhea, malnutrition, dehydration and imbalances of fluids and salts. Specific symptoms and severity of the disease varies from one person to another depending on the age of the patient, the particular segment of small intestine resected, and extent of the resection. In infants, small intestine resection is performed to treat indications such as necrotizing enterocolitis, intestinal anomalies, and midgut volvulus. In older children and adults, Crohn's disease, vascular disease, malignancy, radiation enteritis, trauma, and adhesive obstruction are causative indications associated with resection (Vanderhoof et al., Gastroenterology. 1997; 113(5):1767-1778). Intestinal loss and compromised bowel adaptation leads to inadequate water and nutrient absorption (Schalamon et al., Best Pract Res Clin Gastroenterol 2003; 17(6):931-942). Most patients with SBS experience debilitating diarrhea that severely hinders their health outcomes and quality of life. (Kumpf, JPEN J Parenter Enteral Nutr. 2014; 38(1 Suppl):38S-44S). SBS-associated diarrhea may have several etiologies, therefore management of diarrhea in these patients is challenging and improved therapies are needed. No specific antidiarrheal drug has been studied and approved for SBS associated diarrhea.

SBS refers to the malabsorptive state caused by the physical or functional loss of significant portions of the small intestine. The main causes of SBS in children include congenital or perinatal diseases, such as intestinal atresia (narrowing or absence of a portion of the intestine), abdominal wall defects, malrotation (rotation of the gut outside the abdomen), volvulus (obstruction caused by twisting of the intestine), and long segment Hirschsprung's disease (damaged nerve cells in the colon leading to fecal buildup) (Wales, Seminr Pediair Surg. 2010; 19(1):3-9). However, the most common cause is necrotizing enterocolitis (NEC) accounting for 40-50% of infant SBS cases. (Thompson, Viszeralmedizin. 2014; 30(3):174-178). NEC is a devastating disease that largely affects premature infants where the intestine is invaded by bacteria causing an inflammatory response that ultimately destroys the wall of the intestine. (Terrin et al., Biomed Res Int. 2014; 2014:543765).

SBS is usually defined anatomically as less than 30% of normal intestinal length, which is less than 75 cm in children and less than 200 cm in adults. (Schalamon et al., Best Pract Res Clin Gastroenterol. 2003; 17(6):931-942). The reduction of intestinal length results in a reduced surface area for absorption of nutrients and a more rapid transit of intestinal contents. Poor nutrient processing capability leads to malabsorption-induced diarrhea, dehydration, electrolyte disturbances, and malnutrition. The symptoms and severity of SBS vary markedly and are dependent on the anatomic sections of the intestine that are resected, the length and absorptive capacity of the remaining bowel, and the presence of the active primary disease. (Tappenden, JPEN J Parenter Enteral Nutr. 2014; 38(1 Suppl):14S-22S).

SBS patients are at increased risk for infection and experience impaired wound healing due to malnutrition. (Jeppesen, JPEN J Parenter Enteral Nutr. 2014; 38(1 Suppl):8S-13S). SBS can lead to hypotension and early kidney failure. A number of other chronic complications associated with SBS include hepatic and biliary disease, metabolic bone disease, small bowel bacterial overgrowth, enteric hyperoxaluria, and D-lactic acidosis. (Tappenden et al., JPEN J Parenter Enteral Nutr. 2014; 38(1 Suppl):14S-22S). Therefore, SBS-associated diarrhea and its related complications may be serious and life-threatening.

Based on the diverse etiologies of diarrhea, SBS patients are treated with a variety of antidiarrheal drugs including antimotility drugs, antisecretory agents, antibiotics and probiotics, bile acid-binding resins, and pancreatic enzymes. Management of diarrhea in these patients is challenging, and antidiarrheal treatment regimens must be individualized. In many cases, therapies may only be partially effective, have severe side effects, have potential for addiction, and have contraindications. Regimen optimization and careful consideration of potential drug-drug interactions are necessary (Kumpf, JPEN J Parenter Enteral Nutr. 2014; 38(1 Suppl):38S-44S). The development of a drug for the treatment of SBS with a low potential for drug-drug interactions, effects on drug metabolism, or abuse potential would provide an important benefit for subjects and substantially improve the overall health and quality of life for patients with SBS.

### Congenital Diarrhea Disorders

Congenital Diarrheal Disorders (CDDs) are a group of inherited chronic enteropathies characterized by heterogeneous etiology. (Guarino A et al., Best Pract Res Clin Gastroenterol. 2012; 26(5):649-661). Limited epidemiology studies have been performed on CDDs as the conditions, while severe, are extremely rare. Incidence is determined by experts to be only 200 cases in the United States. (Terrin, G., et al. Int J Mot Sci. 2012; 13(4):4168-4185). Early infancy is the typical age of onset, where the disorders present with severe watery diarrhea, imbalanced serum chemistry and failure to thrive. (Berni Canani R, et al., J Pediatr Gastroenterol Nutr. 2010; 50(4):360-366). Massive dehydration, metabolic acidosis or alkalosis and malnutrition, among other secondary symptoms, expeditiously emerge and become life-threatening. (Overeem A W, et al., Dis Model Mech. 2016; 9(1):1-12; Posovszky C., Best Pract Res Clin Gastroenterol. 2016; 30(2):187-211). Genetically, the autosomal recessive mutations interpose in various genes that quantify each disorder. However, these disorders share a primary common symptom; chronic diarrhea and therefore, the secondary symptoms associated with diarrhea. (Guandalini S, Diarrhea Diagnostic and Therapeutic Advances). Most CDDs are reported to have high mortality rates, and the severity spectrum of the chronic diarrhea is dependent on the disorder and the classification of the defect associated with each. (Field, M., Journal of Clinical Investigation. 2003; 111(7):931-943). Aside from inheritance, no identifiable risk factor has been associated with CDDs.

Early and proper diagnosis, classification and treatment are advantageous to reduce the detrimental disease manifestations. As clinical presentation and pathology may mimic a broad range of conditions, delayed diagnosis is common, leading to high mortality rates in CDD-affected infants. Molecular analysis is a modern diagnostic technique offering superior precision, timely diagnosis and proper classification. Unfortunately, despite early detection and treatment, existing therapy rarely reduces the burden of CDD outcomes. (Field, M., Journal of Clinical Investigation. 2003; 111(7):931-943). Patients receiving the standard treatments, parenteral nutrition (PN), and bowel resection surgery are susceptible to serious complications, adding to the antecedent mortality risk. (Overeem A W, et al., Dis Model Mech. 2016; 9(1):1-12; Posovszky C., Best Pract Res Clin Gastroenterol. 2016; 30(2):187-211). Therefore, new and improved therapy is urgently needed for patients with CDDs to reduce mortality and limit life-long disability. (Guarino A et al., Best Pract Res Clin Gastroenterol. 2012; 26(5): 649-661). No specific antidiarrheal drug has been studied and approved for the treatment of chronic diarrhea in CDD.

Classification of CDDs is utilized in differential diagnosis and depends on the pathophysiological mechanism of the underlying disorder. In the small intestine, nutrients are absorbed from the lumen into the villi via enterocytes (absorptive cells lining the intestinal mucosa), and also play a secretory role. Enterocyte defects underlie the primary diarrhea complications present in all CDDs. The defects are stratified by four different mechanisms: 1) defects of digestion, absorption and transport of nutrients and electrolytes, 2) defects of enterocyte differentiation and polarization, 3) defects of enteroendocrine cells differentiation, and 4) defects of intestinal immune response modulation; and consequently, dysregulate digestion, absorption, and gastrointestinal motility. In addition, defects in innate and adaptive immune responses may involve several types of epithelial cells as well as immune cells of the lamina propria resulting in inflammation and tissue damage.

Microvillus Inclusion Disease (MVID) is a CDD of intestinal epithelial cells. It is caused by a mutation in the MYO5B gene which results in a decrease of absence of myosin Vb function. In cells that line the small intestine, a lack of myosin Vb function changes the cell polarity, which prevents the proper production of structures known as microvilli. Microvilli ordinarily help absorb nutrients from food as it passes through the intestine. Epithelial cells with poorly formed microvilli reduce the intestine's ability to absorb nutrients, leading to chronic diarrhea, malnutrition, and dehydration. The onset of MVID is typically within the first hours to days of life, and affected infants often have difficulty with proper growth (failure to thrive), developmental delay, and liver and kidney problems.

Therefore, diarrhea associated with short bowel syndrome and congenital diarrhea disorders including MVID represent an important and unmet clinical need requiring more effective management.

Delivering and dosing therapeutics to these patient populations has its own unique set of challenges given the age of the population in some circumstances and the anatomical and/or physiological differences in the gastrointestinal tract. Formulations are needed for effective delivery of effective doses to improve management of the conditions for these populations.

### SUMMARY

Disclosed herein are novel formulations comprising lyophilized crofelemer, methods of treating short bowel syndrome (SBS), diarrhea associated SBS, or diarrhea associated with Microvillus Inclusion Disease (MVID), and methods of making novel formulations comprising lyophilized crofelemer.

In one aspect, disclosed are lyophilized pharmaceutical compositions comprising a crofelemer and a pharmaceutically acceptable preserving agent, and optionally, a pharmaceutically acceptable sweetener.

In one aspect, disclosed are liquid pharmaceutical composition comprising crofelemer, wherein the liquid composition is obtained by reconstituting a lyophilized formulation comprising crofelemer.

In one aspect, disclosed are methods of treating short bowel syndrome (SBS), diarrhea associated with SBS, or diarrhea associated with congenital diarrhea disorders selected from Microvillous Inclusion Disease (MVID), Congenital Tufting Enteropathy, Tricho-Hepato-Enteric Syndrome, Immune Dysfunction Polyendocrinopathy, X-linked, IPEX-like Syndrome, Congenital Sodium Diarrhea, Congenital Chloride Diarrhea, Guanylate Cyclase Mutation, and Primary Bile Acid Malabsorption in a subject comprising administering to a subject in need thereof an amount of a pharmaceutical composition comprising a therapeutically effective amount of crofelemer in a lyophilized powder form or a liquid pharmaceutical composition obtained by reconstituting a lyophilized formulation comprising crofelemer.

In one aspect, disclosed are methods of making pharmaceutical compositions comprising crofelemer in a lyophilized form, the method comprising a. mixing crofelemer in water, thereby forming a mixture, and b. lyophilizing the mixture to form a pharmaceutical composition. In a further aspect, lyophilizing the mixture in step b. further comprises i. performing a first freezing step on the mixture, thereby forming a first frozen mixture, ii. annealing the first frozen mixture to yield an annealed mixture, iii. subjecting the annealed mixture to a lowered temperature to maintain the annealed mixture in a frozen state, resulting in a second frozen mixture, iv. drying the second frozen mixture to yield a first dried mixture, v. drying the first dried mixture to yield the pharmaceutical composition, and vi. sealing a container comprising the pharmaceutical composition.

In one aspect, disclosed are methods of making pharmaceutical compositions comprising crofelemer in a lyophilized form, the method comprising a. mixing crofelemer, a pharmaceutically acceptable preserving agent selected from potassium sorbate, sorbic acid, methyl paraben, ethyl paraben, propyl paraben, and sodium benzoate, and a sweetener in water, thereby forming a mixture, and b. lyophilizing the mixture to form a pharmaceutical composition. In a further aspect, lyophilizing mixture in step b. further comprises i. performing a first freezing step on the mixture, thereby forming a first frozen mixture, ii. annealing the first frozen mixture to yield an annealed mixture, iii. subjecting the annealed mixture to a lowered temperature to maintain the annealed mixture in a frozen state, resulting in a second frozen mixture, iv. drying the second frozen mixture to yield a first dried mixture, v. drying the first dried mixture to yield the pharmaceutical composition, and vi. sealing a container comprising the pharmaceutical composition.

In one aspect, disclosed are kits comprising an effective amount of pharmaceutical compositions comprising a therapeutically effective amount of crofelemer in a lyophilized form and one or more of a. instructions for administering the composition in connection with treating short bowel syndrome (SBS) or diarrhea associated therewith SBS and b. instructions for treating short bowel syndrome (SBS) or diarrhea associated therewith.

, In another aspect, disclosed are kits comprising an effective amount of pharmaceutical compositions comprising a therapeutically effective amount of crofelemer in a lyophilized form and one or more of a. instructions for administering the composition in connection with treating MVID or diarrhea associated with MVID and b. instructions for treating MVID or diarrhea associated therewith.

In yet another aspect, disclosed are kits comprising an effective amount of pharmaceutical compositions comprising a therapeutically effective amount of crofelemer in a lyophilized form and one or more of a. instructions for administering the composition in connection with treating Tufting Enteropathy or diarrhea associated with Tufting Enteropathy and b. instructions for treating Tufting Enteropathy or diarrhea associated therewith.

In another aspect, disclosed are kits comprising an effective amount of pharmaceutical compositions comprising a therapeutically effective amount of crofelemer in a lyophilized form and one or more of a. instructions for administering the composition in connection with treating Tricho-Hepato-Enteric Syndrome, Immune Dysfunction Polyendocrinopathy, X-linked, IPEX-like Syndrome, Congenital Sodium Diarrhea, Congenital Chloride Diarrhea, Guanylate Cyclase Mutation, or Primary Bile Acid Malabsorption or diarrhea associated with any one of the foregoing; and b. instructions for treating Tricho-Hepato-Enteric Syndrome, Immune Dysfunction Polyendocrinopathy, X-linked, IPEX-like Syndrome, Congenital Sodium Diarrhea, Congenital Chloride Diarrhea, Guanylate Cyclase Mutation, or Primary Bile Acid Malabsorption or diarrhea associated with any one of the foregoing.

Other embodiments are disclosed infra.

### BRIEF DESCRIPTION OF THE DRAWINGS

The accompanying figure, which is incorporated in and constitutes a part of this specification, illustrates several aspects and together with the description serves to explain the principles of the invention.

FIG. 1 shows a representative plot showing temperature and pressure as a function of time for the lyophilization runs. The representative data shown is for 1.5 g vials.

FIG. 2 shows a representative scheme depicting the manufacturing process flow for crofelemer powder for oral solution, 1.5 g and 450 mg.

FIG. 3 shows a representative scheme depicting a study design schema for safety, tolerability, and efficacy studies of crofelemer in participants with MVID.

### DETAILED DESCRIPTION

### I. Definitions

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a compound” includes a plurality of compounds. Thus, for example, a reference to “a method” includes one or more methods, and/or steps of the type described herein and/or which will become apparent to those persons skilled in the art upon reading this disclosure.

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with crofelemer, where the untreated subjects have, or are subject to developing, the same or similar disease, condition, symptom or the like. Amelioration of a disease, condition, symptom or assay parameter may be determined subjectively or objectively, e.g., self-assessment by a subject(s), by a clinician's assessment or by conducting an appropriate assay or measurement. Amelioration may be transient, prolonged or permanent or it may be variable at relevant times during or after crofelemer is administered to a subject or is used in an assay or other method described herein or a cited reference, e.g., within timeframes described infra, or about 1 hour after the administration or use of crofelemer to about 7 days, 2 weeks, 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with crofelemer, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively. Modulation may be transient, prolonged or permanent or it may be variable at relevant times during or after crofelemer is administered to a subject e.g., within times descried herein, or within about 1 hour of the administration or use of crofelemer to about 1 day, two days, three days, four days, 1 week, 2 weeks, 28 days, or 3 months or more after a subject(s) has received crofelemer.

As used herein, “subject” includes an animal, including a person, such as adult or pediatric persons.

The language “a therapeutically effective amount” of a compound refers to an amount of an active ingredient which is effective, within a single or multiple dose administration to treat the subject for a particular condition.

The language “a prophylactically effective amount” of a compound refers to an amount of an active ingredient which is effective within single or multiple dose to prevent or delay onset of symptoms in the subject.

The term “administration” or “administering” includes routes of introducing an active ingredient to a subject to perform its intended function. Examples of routes of administration that may be used include gastric feeding tube, duodenal feeding tube, oral or rectal. The pharmaceutical preparations may be given by forms described herein.

Administration “in combination with” one or more further therapeutic agents includes simultaneous (concurrent) and consecutive administration in any order.

The phrase “pharmaceutically acceptable” refers to a quality of being suitable for use in contact with the tissues of human beings and animals without excessive toxicity, irritation, allergic response, or other problem or complication, commensurate with a reasonable benefit/risk ratio.

The phrase “carrier” includes material, composition or vehicle, such as a liquid or solid filler, diluent, excipient, solvent or encapsulating material, involved in carrying or transporting the subject chemical from one organ, or portion of the body, to another organ, or portion of the body.

The term “treat” or “treatment” as used herein is intended to include the reduction or amelioration of the progression, severity, and/or duration of a condition or one or more symptoms of a condition. For example, treating SBS or MVID or Tufting Enteropathy may include an improvement of the following symptoms of SBS or MVID or Tufting Enteropathy, including, for example, a decrease in an amount of parenteral nutrition needed by the patient, a decrease in the number of bowel movements per day (daily frequency of stools), a decrease in the number of watery bowel movements per day (daily frequency of abnormal stools), a decrease in symptom frequency (urgency, fecal incontinence), a decrease in symptom severity (abdominal pain or discomfort), a decrease in daily stool consistency score (watery to formed), a decrease in stool consistency leading to formed stools from watery stools, improvement in nutritional status, hydration and/or electrolyte balance, improvement in hepatic or biliary disease, or reduction in morbidity or risk of death. Such improvements are based on a comparison of the symptom prior to treatment.

The term “obtaining” is intended to include purchasing, synthesizing, isolating, extracting or otherwise acquiring.

### II. Active Compounds

### A. Crofelemer

Crofelemer is an oligomeric proanthocyanidin extracted and purified from the red, viscous latex of the plant Croton lechleri of the family Euphorbiaceae. The plant is widely distributed throughout tropical Central America and South America and is widely recognized by ethnobotanists and local healers for its medicinal properties (McRae 1988), including for the treatment of diarrhea. Crofelemer is believed to exert its anti-diarrheal effect through partial inhibition and/or modulation of CFTR (cystic fibrosis transmembrane conductance regulator) chloride (Cl—) channel. Crofelemer has demonstrated in vitro activity against cholera toxin, forskolin, E. coli LT and STa toxin-mediated Cl— secretion, and to normalize electrolyte and fluid accumulation in CT-treated mice (Gabriel 1999, Fischer 2004, Adam 2005) via its effects on the CFTR chloride channel. Crofelemer also significantly improved the secretory diarrhea in humans due to enterotoxigenic E. coli (DiCesare 2002), which is also thought to evoke secretory diarrhea through activation of CFTR (Kunzelmann 2002). Blockade or inhibitory modulation of the CFTR channel could be anticipated to have negative consequences in man, even mimicking cystic fibrosis. However, crofelemer has virtually no systemic bioavailability in humans. When studied, the results indicated that there was little or no absorption of crofelemer from the GI tract, and that crofelemer was well tolerated by normal male subjects. Thus, the site of action of crofelemer is topical in the gastrointestinal tract.

Crofelemer (CAS 148465-45-6) is an oligomeric proanthocyanidin of varying chain lengths derived from the Dragon's Blood Croton lecheri of the family Euphorbiaceae. Crofelemer has an average molecular weight ranging between approximately 1500 daltons and approximately 2900 daltons. The monomers comprising crofelemer comprise catechin, epicatechin, gallocatechin, and epigallocatechin. The chain length of crofelemer ranges from about 3 to about 30 units with an average chain length of about 7-8 units. The structure of crofelemer is shown below.

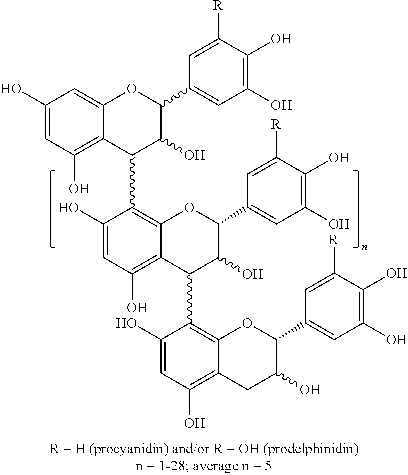

Wherein the average n=1-28.

Another method for isolating crofelemer can be found in U.S. Patent Publication No. 2005/0019389, the contents of which are expressly incorporated herein.

Examples of the preparation and use of crofelemer have been described in U.S. Pat. No. 7,556,831, US Patent Publication 20070254050 and US Patent Publication 20080031984, all of which are incorporated herein by reference in their entirety.

### III. Pharmaceutical Compositions

Pharmaceutical compositions described hereafter comprises crofelemer and a pharmaceutically acceptable preserving agent in a powder form or a form obtained from lyophilization. In a further embodiment, such pharmaceutical compositions comprise a therapeutically effective amount of crofelemer to treat SBS, MVID, or Tufted Enteropathy or diarrhea associated any one of the foregoing as described herein.

Embodiments of pharmaceutical compositions can be liquid pharmaceutical composition, wherein the liquid composition is obtained by reconstituting the pharmaceutical composition as described herein, such as a lyophilized composition or a composition in powder form. Such pharmaceutical compositions can be reconstituted with a pharmaceutically acceptable solvent or buffer or water.

As mentioned above, the pharmaceutical composition further comprises a pharmaceutically acceptable preserving agent. A pharmaceutically acceptable preserving agent is an agent that extends the shelf life of a pharmaceutical composition by inhibiting the growth of potentially harmful microorganisms. In some embodiments, the preserving agent is selected from potassium sorbate, sorbic acid, methyl paraben, ethyl paraben, propyl paraben, and sodium benzoate. In particular embodiments, the pharmaceutically acceptable preserving agent is sodium benzoate.

The pharmaceutically acceptable preserving agent can be added in any form, such as a solution of the preserving agent or a solid form of the preserving agent, such as a pharmaceutically acceptable salt or as a powder, such one obtained via lyophilization.

In embodiments, the pharmaceutical composition further comprises a sweetener. A sweetener imparts a sweet taste to a formulation. By imparting a sweet taste, the sweetener can improve the palatability and acceptability of a pharmaceutical composition. The sweetener may be any sweetening agent conventionally known in the art for use in pharmaceutical compositions. Examples of suitable sweeteners include, but are not limited to, sucralose, sucrose, fructose, glucose, erythritol, martitol, lactitol, sorbitol, mannitol, xylitol, tagatos, trehalose, galactose, ramnorth, ribulose, treose, arabinose, xylose, liquisource, allose, altrose, mannose, idose, lactose, maltose, converted sugar, isotorehalose, neotrehalose, acesulfame potassium, acesulfame acid, aspartame, alitame, saccharin, neohesperidin dihydrocalcone, steviol glycosides, and cyclamate. In particular embodiments, the sweetener is sucralose.

The sweetener can be added in any form, such as a solution of the sweetener or a solid form of the sweetener, such as a pharmaceutically acceptable salt or as a powder, such as one obtained by lyophilization.

In embodiments, the pharmaceutical composition further comprises one or more buffering agents configured to buffer a solution between a pH of about 3.5 and about 5. A pH between about 3.5 and about 5 is desired particularly for embodiments in which the pharmaceutical composition further comprises sodium benzoate as a pharmaceutically acceptable preserving agent. Such a pH range offers solution stability and the preserving capability for sodium benzoate. The one or more buffering agents may be any pharmaceutically acceptable buffering agent capable of buffering a solution between a pH of about 3.5 and about 5. In particular embodiments, the buffering agents can be an organic acid. In certain embodiments, the buffering agent can be selected from citric acid, citrate, and acetate.

The one or more buffering agents can be added in any form, such as a solution of the buffering agent or a solid form of the buffering agent, such as a pharmaceutically acceptable salt or as a powder, such as one obtained by lyophilization.

In embodiments, the pharmaceutical composition further comprises a bulking agent. A bulking agent can improve stability of the pharmaceutical composition as a “cake” formed during the lyophilization process. A bulking agent may provide structure to the lyophilized cake, preventing its collapse and destabilization. Examples of suitable bulking agents may include, but are not limited to, mannitol, glycine, sucrose, raffinose, hydroxyethyl starch (HES), dextran, polyvinylpyrolidone, carboxymethylcellulose, lactose, sorbitol, trehalose, and xylitol. In certain embodiments, the bulking agent is mannitol.

The bulking agent can be added in any form, such as a solution of the bulking agent or a solid form of the bulking agent, such as a pharmaceutically acceptable salt or as a powder, such as one obtained by lyophilization.

In one aspect, the pharmaceutical composition is formulated to be administered orally or via a gastric feeding tube or duodenal feeding tube. In preferred embodiments, the pharmaceutical composition is administered as a liquid oral dosage form.

The pharmaceutical compositions described herein can comprise additional excipients, for example, one or more of a diluting agent, binding agent, lubricating agent, disintegrating agent, coloring agent, or flavoring agent.

In certain embodiments, the pharmaceutical composition can comprise a mass ratio of crofelemer to sodium benzoate of about 300:1 to about 13:1. For example, the pharmaceutical composition can comprise a mass ratio of crofelemer to sodium benzoate of about 300:1 to about 25:1, about 300:1 to about 50:1, about 300:1 to about 100:1, about 250:1 to about 25:1, about 250:1 to about 50:1, about 250:1 to about 100:1, about 200:1 to about 25:1, about 200:1 to about 50:1, about 200:1 to about 100:1, about 150:1 to about 25:1, about 150:1 to about 50:1, about 150:1 to about 100:1, about 100:1 to about 25:1, about 100:1 to about 50:1, about 80:1 to about 75:1, about 80:1 to about 65:1, about 80:1 to about 55:1, about 80:1 to about 45:1, about 80:1 to about 35:1, about 80:1 to about 25:1, about 80:1 to about 15:1, about 80:1 to about 14:1, about 80:1 to about 13:1, about 75:1 to about 13:1, about 65:1 to about 13.1, about 55:1 to about 13.1, about 45:1 to about 13.1, about 35:1 to about 13.1, about 30:1 to about 13.1, about 65:1 to about 15:1, about 55:1 to about 20:1, or about 45:1 to about 25:1.

In some aspects, the pharmaceutical composition can comprise a mass ratio of crofelemer to sucralose of from about 10:1 to about 5:1. For example, the pharmaceutical composition can comprise a mass ratio of crofelemer to sucralose of from about 10:1 to about 6:1, of about 10:1 to about 7:1, of about 10:1 to about 8:1, of about 10:1 to about 9:1, of about 9:1 to about 5:1, of about 9:1 to about 6:1, of about 9:1 to about 7:1, of about 9:1 to about 8:1, of about 8:1 to about 5:1, of about 8:1 to about 6:1, of about 8:1 to about 7:1, of about 7:1 to about 5:1, of about 7:1 to about 6:1, or of about 6:1 to about 5:1.

In certain embodiments, the pharmaceutical composition can comprise a mass ratio of crofelemer to citric acid of from about 90:1 to about 75:1. For example, the pharmaceutical composition can comprise a mass ratio of crofelemer to citric acid of from about 90:1 to about 80:1, about 90:1 to about 85:1, about 85:1 to about 75:1, or about 80:1 to about 75:1.

In certain embodiments, the pharmaceutical composition can comprise a mass ratio of crofelemer to citrate of from about 100:1 to about 80:1. For example, the pharmaceutical composition can comprise a mass ratio of crofelemer to citrate of from about 100:1 to about 95:1, about 100:1 to about 90:1, about 100:1 to about 85:1, about 95:1 to about 80:1, about 95:1 to about 85:1, about 95:1 to about 90:1, about 90:1 to about to about 80:1, or about 90:1 to about 85:1.

In particular embodiments, the pharmaceutical composition can comprise a mass ratio of crofelemer to mannitol of from about 3:1 to about 1:0.5. For example, the pharmaceutical composition can comprise a mass ratio of crofelemer to mannitol of from about 3:1 to about 1:1, about 3:1 to 1.5, about 3:1 to 1:2, about 3:1 to 1:2.5, about 2.5:1 to about 1:1, about 2.5:1 to about 1:1.5, about 2.5 to about 2, or about 2:1 to about 1:0.5.

In some aspects, the pharmaceutical composition can comprise from about 0 to about 10% moisture content by weight. For example, the pharmaceutical composition can comprise from about 0 to about 9%, about 0 to about 8%, about 0 to about 7%, about 0 to about 6%, about 0 to about 5%, about 0 to about 4%, about 0 to about 3%, about 0 to about 2%, about 0 to about 1.5%, about 0 to about 1%, about 0 to about 0.5%, about 0.5 to about 2.5%, about 0.5% to about 2%, about 0.5% to about 1.5%, about 0.5% to about 1%, about 1% to about 2.5%, about 1% to about 2%, about 1% to about 1.5%, about 1% to about 5%, about 1% to about 7%, about 5% to about 10%, or about 2 to about 2.5% moisture content by weight.

In particular embodiments, the pharmaceutical composition comprises sodium benzoate in a mass ratio of crofelemer to sodium benzoate from about 300:1 to about 13:1, sucralose in a mass ratio of crofelemer to sucralose from about 10:1 to about 5:1, citric acid in a mass ratio of crofelemer to citric acid from about 90:1 to about 75:1, citrate in a mass ratio of crofelemer to citrate from about 100:1 to about 80:1, and mannitol in a mass ratio of crofelemer to mannitol from about 3:1 to about 1:0.5, further wherein the sodium benzoate, sucralose, citric acid, citrate, and mannitol are in a powder form or a form obtained from lyophilization.

In some aspects, the liquid pharmaceutical composition comprises a concentration of crofelemer from about 2 mg/mL to about 120 mg/mL. For example, the liquid pharmaceutical composition can comprise from about 20 mg/mL to about 110 mg/mL, about 20 mg/mL to about 100 mg/mL, about 20 mg/mL to about 90 mg/mL, about 20 mg/mL to about 80 mg/mL, about 20 mg/mL to about 70 mg/mL, about 20 mg/mL to about 60 mg/mL, about 20 mg/mL to about 50 mg/mL, about 20 mg/mL to about 40 mg/mL, about 20 mg/mL to about 30 mg/mL, about 30 mg/mL to about 120 mg/mL, about 40 mg/mL to about 120 mg/mL, about 50 mg/mL to about 120 mg/mL, about 60 mg/mL to about 120 mg/mL, about 70 mg/mL to about 120 mg/mL, about 80 mg/mL to about 120 mg/mL, about 90 mg/mL to about 120 mg/mL, about 100 mg/mL to about 120 mg/mL, about 110 mg/mL, about 40 mg/mL to about 110 mg/mL, about 50 mg/mL to about 90 mg/mL, about 2 mg/mL to about 100 mg/mL, about 5 mg/mL to about 100 mg/mL, about 15 mg/mL to about 100 mg/mL, about 5 mg/mL to about 90 mg/mL, or about 60 mg/mL to about 85 mg/mL of crofelemer.

In some aspects, the liquid pharmaceutical is stable at room temperature for 7, 10, 12, 14, 16, 20, or 30 days.

### IV. Methods of Making Pharmaceutical Compositions

Provided herein are methods of making pharmaceutical compositions comprising a therapeutically effective amount of crofelemer in a lyophilized powder form. Also provided herein are methods of making pharmaceutical compositions comprising a therapeutically effective amount of crofelemer in a lyophilized powder form and a pharmaceutically acceptable preserving agent.

The process of lyophilization allows for the formation of a more stable pharmaceutical composition through a commercially validated method. By controlling the pressure and temperature within the lyophilizer, liquid is removed from the pharmaceutical composition, resulting in a “cake”-like solid that exhibits greater stability compared to the initial aqueous solution. This allows for longer shelf times in which the pharmaceutical composition can be stored in a wider range of conditions compared to its aqueous form. The lyophilized pharmaceutical composition comprises a high surface area, enabling the composition to be reconstituted quickly to yield a liquid composition.

In one embodiment, the method comprises a. mixing crofelemer in water, thereby forming a mixture, and b. lyophilizing the mixture to form a pharmaceutical composition. In a further aspect, a pharmaceutically acceptable preserving agent selected from potassium sorbate, sorbic acid, methyl paraben, ethyl paraben, propyl paraben, and sodium benzoate and a sweetener are added after step b. In a still further aspect, the pharmaceutically acceptable preserving agent is sodium benzoate.

In another embodiment, the method comprises a. mixing crofelemer, a pharmaceutically acceptable preserving agent selected from potassium sorbate, sorbic acid, methyl paraben, ethyl paraben, propyl paraben, and sodium benzoate, and a sweetener in water, thereby forming a mixture, and b. lyophilizing the mixture to form a pharmaceutical composition.

In embodiments, lyophilizing the mixture in step b. further comprises i. performing a first freezing step on the mixture, thereby forming a first frozen mixture, ii. annealing the first frozen mixture to yield an annealed mixture, iii. subjecting the annealed mixture to a lowered temperature to maintain the annealed mixture in a frozen state, resulting in a second frozen mixture, iv. a primary drying step comprising drying the frozen mixture to yield a first dried mixture, v. a secondary drying step comprising drying the first dried mixture to yield the pharmaceutical composition, and vi. sealing a container comprising the pharmaceutical composition.

In embodiments, a first freezing step is performed on the mixture to form a first frozen mixture. During the first freezing step, the temperature is lowered such that the mixture is cooled below its critical temperature (T_{crit}) to ensure that it is fully frozen. T_{crit} of the pharmaceutical composition is affected by the combination and proportions of the components within the mixture. The rate at which the temperature is lowered affects the structure of the ice matrix, which has an impact on the ease of flow of the sublimated vapor out of the sample.

In embodiments, the first freezing step is performed at a temperature of from about −80° C. to about −10° C. The first freezing step can be performed, for example, at a temperature of from about −80° C. to about −20° C., −70° C. to about −15° C., −60° C. to about −30° C., about −60° C. to about −40° C., about −60° C. to about −50° C., about −50° C. to about −20° C., about −50° C. to about −30° C., about −50° C. to about −40° C., or about −40° C. to about −30° C.

In embodiments, the first freezing step is performed at a temperature ramping rate of about −10° C./hour to about −60° C./hour. The first freezing step can be performed, for example, at a temperature ramping rate of about −10° C./hour to about −55° C./hour, about −10° C./hour to about −50° C./hour, about −10° C./hour to about −45° C./hour, about −20° C./hour to about −60° C./hour, about −40° C./hour to about −25° C./hour, about −40° C./hour to about −30° C./hour, about −40° C./hour to about −35° C./hour, about −35° C./hour to about −25° C./hour, or about −30° C./hour to about −20° C./hour.

In embodiments, the first freezing step comprises a soak time of about 1 to about 6 hours. The first freezing step can comprise, for example, a soak time of from about 1 to about 5 hours, about 1 to about 4 hours, about 2 to about 5 hours, about 2 to about 3.5 hours, about 2 to about 3 hours, about 2.5 to about 4 hours, about 2.5 to about 3.5 hours, about 3 to about 4 hours, or about 3.5 to about 4 hours.

In embodiments, the first freezing step, annealing, and subjecting the annealed mixture to a lowered temperature are performed at a chamber pressure of about ≤20 PSIA.

In embodiments, an annealing step is performed on the first frozen mixture to yield an annealed mixture. An annealing step may be performed, for example, when the pharmaceutical composition comprises a bulking agent, such as mannitol. Bulking agents such as mannitol can form a metastable glass with incomplete crystallization during the first freezing step. During the annealing step, the temperature is cycled to obtain more complete crystallization. The annealing step can also provide the benefits of larger crystal growth and shorter drying times during the drying steps. During the annealing step, the first frozen mixture remains frozen.

In embodiments, the annealing is performed at a temperature of from about 0° C. to about −40° C. The annealing can be performed, for example, at a temperature of from about 0 to about −35° C., about −5° C. to about −20° C., about −5° C. to about −15° C., about −5° C. to about −10° C., about −10° C. to about −25° C., about −10° C. to about −20° C. about −15° C. to about −25° C., or about −20° C. to about −25° C.

In embodiments, the annealing is performed at a temperature ramping rate of about −5° C./hour to about −50° C./hour. The annealing can be performed, for example, at a temperature ramping rate of about −5° C./hour to about −40° C./hour, about −10° C./hour to about −40° C./hour, about −20° C./hour to about −35° C./hour, about −20° C./hour to about −30° C./hour, about −20° C./hour to about −25° C./hour, about −25° C./hour to about −40° C./hour, about −25° C./hour to about −35° C./hour, about −20° C./hour to about −30° C./hour, about −30° C./hour to about −40° C./hour, or about −35° C./hour to about −40° C./hour.

In embodiments, the annealing comprises a soak time of about 2 to about 10 hours. The annealing can comprise, for example, a soak time of from about 2 to about 9 hours, about 3 to about 8 hours, about 4 to about 7 hours, about 4 to about 6 hours, about 4 to about 5 hours, about 5 to about 8 hours, about 5 to about 7 hours, about 5 to about 6 hours, about 6 to about 8 hours, or about 6 to about 7 hours.

In embodiments, subjecting the annealed mixture to a lowered temperature to maintain the annealed mixture in a frozen state, resulting in a second frozen mixture. During subjecting the annealed mixture to a lowered temperature, the temperature is again lowered such that the annealed mixture is cooled below its critical temperature (T_{crit}) to ensure that it remains frozen. T_{crit} of the pharmaceutical composition is affected by the combination and proportions of the components within the mixture. The rate at which the temperature is lowered affects the structure of the ice matrix, which has an impact on the ease of flow of the sublimated vapor out of the sample. Subjecting the annealed mixture to a lowered temperature can be performed, for example, when an annealing step has been performed.

In embodiments, subjecting the annealed mixture to a lowered temperature is performed at a temperature of from about −80° C. to about −10° C. The first second step can be performed, for example, at a temperature of from about −80° C. to about −15° C., about −70° C. to about −20° C., about −60° C. to about −30° C., about −60° C. to about −40° C., about −60° C. to about −50° C., about −50° C. to about −20° C., about −50° C. to about −30° C., about −50° C. to about −40° C., or about −40° C. to about −30° C.

In embodiments, subjecting the annealed mixture to a lowered temperature is performed at a temperature ramping rate of about −10° C./hour to about −60° C./hour. Subjecting the annealed mixture to a lowered temperature can be performed, for example, at a temperature ramping rate of about −10° C./hour to about −55° C./hour, about −10° C./hour to about −50° C./hour, about −10° C./hour to about −45° C./hour, about −20° C./hour to about −60° C./hour, about −40° C./hour to about −25° C./hour, about −40° C./hour to about −30° C./hour, about −40° C./hour to about −35° C./hour, about −35° C./hour to about −25° C./hour, or about −30° C./hour to about −20° C./hour.

In embodiments, subjecting the annealed mixture to a lowered temperature comprises a soak time of about 1 to about 10 hours. Subjecting the annealed mixture to a lowered temperature can comprise, for example, a soak time of about 1 to about 9 hours, about 1 to about 8 hours, about 2 to about 9 hours, about 2 to about 7 hours, about 2 to about 5 hours, about 2 to about 4 hours, about 2 to about 3 hours, about 3 to about 6 hours, about 3 to about 5 hours, or about 4 to about 5 hours.

In embodiments, a primary drying step is performed on the second frozen mixture to yield a first dried mixture. During the primary drying step, the second frozen mixture is initially dried by sublimation. The pressure of the chamber is reduced to a very low level, while the temperature is simultaneously raised slightly to allow the solvent to sublime. During this step, the temperature must be kept below T_{crit} to prevent the mixture from melting or structural collapse.

In embodiments, the primary drying step is performed at a temperature of from about 0° C. to about 50° C. The primary drying step can be performed, for example, at a temperature of from about 0° C. to about 40° C., about 10° C. to about 40° C., about 10° C. to about 30° C., about 15° C. to about 30° C., about 15° C. to about 25° C., about 15° C. to about 20° C., about 20° C. to about 35° C., about 20° C. to about 30° C., about 25° C. to about 35° C., or about 30° C. to about 35° C.

In embodiments, the primary drying step is performed at a temperature ramping rate of about 10° C./hour to about 50° C./hour. The primary drying step can be performed, for example, at a temperature ramping rate of about 10° C./hour to about 40° C./hour, about 20° C./hour to about 35° C./hour, about 20° C./hour to about 30° C./hour, about 25° C./hour to about 40° C./hour, about 25° C./hour to about 35° C./hour, or about 30° C./hour to about 35° C./hour.

In embodiments, the primary drying step comprises a soak time of about 15 to about 50 hours. The primary drying step can comprise, for example, a soak time of about 15 to about 45 hours, about 15 to about 40 hours, about 20 to about 35 hours, about 20 to about 30 hours, about 25 to about 40 hours, about 25 to about 35 hours, about 25 to about 30 hours, or about 30 to about 40 hours.

In embodiments, the primary drying step is performed at a chamber pressure of about 10 microns to about 100 microns. The primary drying step can be performed, for example, at a chamber pressure of about 10 microns to about 90 microns, about 10 microns to about 80 microns, about 20 microns to about 90 microns, about 30 microns to about 90 microns, about 40 microns to about 70 microns, about 40 microns to about 60 microns, about 50 microns to about 80 microns, about 50 microns to about 70 microns, or about 60 microns to about 80 microns.

In embodiments, a secondary drying step is performed on first dried mixture to yield the pharmaceutical composition. During the secondary drying step, any solvent that remains chemically bound to the mixture is removed via desorption. During this process, the temperature is raised and the pressure is reduced to a minimum.

In embodiments, the secondary drying step is performed at a temperature of from about 0° C. to about 50° C. The secondary drying step can be performed, for example, at a temperature of from about 0° C. to about 40° C., about 10° C. to about 40° C., about 10° C. to about 30° C., about 15° C. to about 30° C., about 15° C. to about 25° C., about 15° C. to about 20° C., about 20° C. to about 35° C., about 20° C. to about 30° C., about 25° C. to about 35° C., or about 30° C. to about 35 CC.

In embodiments, the secondary drying step comprises a soak time of about 5 to about 40 hours. The secondary drying step can comprise, for example, a soak time of about 5 to about 35 hours, about 5 to about 30 hours, about 10 to about 25 hours, about 10 to about 20 hours, about 15 to about 30 hours, about 15 to about 25 hours, about 15 to about 20 hours, or about 20 to about 30 hours.

In embodiments, the secondary drying step is performed at a chamber pressure of about 200 microns to about 1000 microns. The secondary drying step can be performed, for example, at a chamber pressure of about 200 microns to about 900 microns, about 200 microns to about 800 microns, about 300 microns to about 900 microns, about 400 microns to about 900 microns, about 400 microns to about 700 microns, about 400 microns to about 600 microns, about 500 microns to about 800 microns, about 500 microns to about 700 microns, or about 600 microns to about 800 microns.

In embodiments, sealing a container comprising the pharmaceutical composition is performed at a temperature of about 5° C. to about 40° C. Sealing a container comprising the pharmaceutical composition can be performed at, for example, a temperature of from about 5° C. to about 30° C., about 10° C. to about 30° C., about 15° C. to about 30° C., about 15° C. to about 25° C., about 15° C. to about 20° C., about 20° C. to about 35° C., about 20° C. to about 30° C., about 25° C. to about 35° C., or about 30° C. to about 35° C.

In embodiments, sealing a container comprising the pharmaceutical composition is performed at chamber pressure of about 0 PSIA to about 15 PSIA. Sealing a container comprising the pharmaceutical composition can be performed at, for example, a pressure of from about 0 PSIA to about 13 PSIA, about 0 to about 10 PSIA, about 3 PSIA to about 15 PSIA, about 5 PSIA to about 15 PSIA, about 5 PSIA to about 9 PSIA, about 5 PSIA to about 8 PSIA, about 5 PSIA to about 7 PSIA, about 6 PSIA to about 10 PSIA, about 7 PSIA to about 10 PSIA, or about 8 PSIA to about 10 PSIA.

In embodiments, the pharmaceutically acceptable preserving agent is sodium benzoate. In a further embodiment, the mass ratio of crofelemer to sodium benzoate is from about 300:1 to about 13:1. For example, the mass ratio of crofelemer to sodium benzoate can be from 300:1 to about 25:1, about 300:1 to about 50:1, about 300:1 to about 100:1, about 250:1 to about 25:1, about 250:1 to about 50:1, about 250:1 to about 100:1, about 200:1 to about 25:1, about 200:1 to about 50:1, about 200:1 to about 100:1, about 150:1 to about 25:1, about 150:1 to about 50:1, about 150:1 to about 100:1, about 100:1 to about 25:1, about 100:1 to about 50:1, about 80:1 to about 75:1, about 80:1 to about 65:1, about 80:1 to about 55:1, about 80:1 to about 45:1, about 80:1 to about 35:1, about 80:1 to about 25:1, about 80:1 to about 15:1, about 80:1 to about 14:1, about 80:1 to about 13:1, about 75:1 to about 13:1, about 65:1 to about 13.1, about 55:1 to about 13.1, about 45:1 to about 13.1, about 35:1 to about 13.1, about 30:1 to about 13.1, about 65:1 to about 15:1, about 55:1 to about 20:1, or about 45:1 to about 25:1.

In embodiments, the sweetener is selected from sucralose, sucrose, fructose, glucose, erythritol, martitol, lactitol, sorbitol, mannitol, xylitol, tagatos, trehalose, galactose, ramnorth, ribulose, treose, arabinose, xylose, liquisource, allose, altrose, mannose, idose, lactose, maltose, converted sugar, isotorehalose, neotrehalose, acesulfame potassium, acesulfame acid, aspartame, alitame, saccharin, steviol glycoside(s), neohesperidin dihydrocalcone, and cyclamate. In a preferred embodiment, the sweetener is sucralose.

In embodiments, the method can comprise a mass ratio of crofelemer to sucralose of from about 10:1 to about 5:1. For example, the method can comprise a mass ratio of crofelemer to sucralose of from about 10:1 to about 6:1, of about 10:1 to about 7:1, of about 10:1 to about 8:1, of about 10:1 to about 9:1, of about 9:1 to about 5:1, of about 9:1 to about 6:1, of about 9:1 to about 7:1, of about 9:1 to about 8:1, of about 8:1 to about 5:1, of about 8:1 to about 6:1, of about 8:1 to about 7:1, of about 7:1 to about 5:1, of about 7:1 to about 6:1, or of about 6:1 to about 5:1.

In embodiments, the method comprises addition of citric acid. In a further embodiment, the method can comprise a mass ratio of crofelemer to citric acid of from about 90:1 to about 75:1. For example, the method can comprise a mass ratio of crofelemer to citric acid of from about 90:1 to about 80:1, about 90:1 to about 85:1, about 85:1 to about 75:1, or about 80:1 to about 75:1.

In embodiments, the method comprises addition of citrate. In various embodiments, the method can comprise a mass ratio of crofelemer to citrate of from about 100:1 to about 80:1. For example, the method can comprise a mass ratio of crofelemer to citrate of from about 100:1 to about 95:1, about 100:1 to about 90:1, about 100:1 to about 85:1, about 95:1 to about 80:1, about 95:1 to about 85:1, about 95:1 to about 90:1, about 90:1 to about to about 80:1, or about 90:1 to about 85:1.

In embodiments, the method comprises addition of mannitol. In particular embodiments, the method can comprise a mass ratio of crofelemer to mannitol of from about 3:1 to about 1:0.5. For example, the pharmaceutical composition can comprise a mass ratio of crofelemer to mannitol of from about 3:1 to about 1:1, about 3:1 to 1.5, about 3:1 to 1:2, about 3:1 to 1:2.5, about 2.5:1 to about 1:1, about 2.5:1 to about 1:1.5, about 2.5 to about 2, or about 2:1 to about 1:0.5.

In one embodiment, the method comprises a. mixing crofelemer, sodium benzoate in a mass ratio of crofelemer to sodium benzoate from about 300:1 to about 13:1, sucralose in a mass ratio of crofelemer to sucralose from about 10:1 to about 5:1, citric acid in a mass ratio of crofelemer to citric acid from about 90:1 to about 75:1, citrate in a mass ratio of crofelemer to citrate from about 100:1 to about 80:1, and mannitol in a mass ratio of crofelemer to mannitol from about 3:1 to about 1:0.5, thereby forming a mixture, and b. lyophilizing the mixture to form a pharmaceutical composition, wherein lyophilizing the mixture comprises: i. performing a first freezing step on the mixture, wherein the first freezing step is performed at a temperature of from about −50° C. to about −30° C. at a temperature ramping rate of about −10° C./hour to about −60° C./hour for a soak time of about 2 to about 4 hours at a chamber pressure of about ≤12 PSIA, thereby forming a first frozen mixture, ii. annealing the first frozen mixture, wherein annealing is performed at a temperature of from about −10° C. to about −20° C. at a temperature ramping rate of about −20° C./hour to about −40° C./hour for a soak time of about 4 to about 8 hours at a chamber pressure of about ≤12 PSIA, thereby yielding an annealed mixture, iii. subjecting the annealed mixture to a lowered temperature to maintain the annealed mixture in a frozen state, resulting in a second frozen mixture and is performed at a temperature of from about −50° C. to about −30° C. at a temperature ramping rate of about −20° C./hour to about −40° C./hour for a soak time of about 2 to about 6 hours at a chamber pressure of about ≤12 PSIA, thereby forming a second frozen mixture, iv. a primary drying step comprising drying the second frozen mixture, wherein the primary drying step is performed at a temperature of about 20° C. to about 30° C. at a temperature ramping rate of about 20° C./hour to about 40° C./hour for a soak time of about 20 to about 40 hours at a chamber pressure of about 40 microns to about 80 microns, thereby yielding a first dried mixture, v. a secondary drying step comprising drying the first dried mixture, wherein the secondary drying step is performed at a temperature of about 20° C. to about 30° C. for a soak time of about 10 to about 30 hours at a chamber pressure of about 400 microns to about 800 microns to yield the pharmaceutical composition, and vi. sealing a container comprising the pharmaceutical composition, wherein sealing a container is performed at about 15° C. to about 35° C. at a chamber pressure of about 5 PSIA to about 10 PSIA.

### V. Methods of Treating SBS and Diarrhea Associated with SBS

Patients suffering from SBS have reduced surface area for absorption of nutrients and a more rapid transit of intestinal contents. Many SBS patients require long term parenteral nutrition and may experience serious metabolic complications, including hepatic and biliary disorders, increased risk of infection and other serious, chronic complications. Diarrhea in SBS patients may be caused by a number of SBS-related stimuli, such as bacterial infection, dihydroxy bile acids, hydroxylated fatty acids, or inflammatory mediators. Anti-motility and acid-suppressing therapies can cause bacterial overgrowth, promoting secretory diarrhea. (Kumpf, JPEN J Parenter Enteral Nutr. 2014; 38(1 Suppl.):38S-44S). SBS patients are treated with a variety of anti-diarrhea drugs, including anti-motility drugs, anti-secretory agents, and antibiotics. In all aspects, SBS is a life-threatening condition with high chance of mortality or life-long morbidity. Provided herein are methods of addressing the diarrhea associated with SBS that improve the nutritional status, hydration, electrolyte balance, health, quality of life and prognosis for SBS patients.

A method in accordance with the present disclosure involves treating short bowel syndrome (SBS) or diarrhea associated with SBS in a subject by administering to the subject in need thereof a therapeutically effective amount of the pharmaceutical compositions disclosed herein. The subject is preferably a human. The pharmaceutical composition may be a reconstituted liquid formulation, including, in an embodiment, at a concentration of about 2 mg/mL to about 120 mg/mL, reconstituted from a powder or dispersible form of crofelemer, including, in an embodiment, a lyophilized form of crofelemer.

In some embodiments, the subject with SBS to be treated has small intestinal bacterial overgrowth, radiation enteropathy, celiac disease, cystic fibrosis, or chronic pancreatitis. In certain embodiments, the subject had a vagotomy, cholecystectomy, duodenal resection, ileal resection, or a jejunal resection. In certain embodiments, the subject with SBS has intestinal failure. In certain embodiments, the subject with SBS had a small intestine resection as a result of treatment for Crohn's disease, vascular disease, malignancy, radiation enteritis, trauma, or adhesive obstruction. In some embodiments, the subject with SBS does not have inflammatory bowel disease, inflammatory bowel syndrome, or Crohn's disease. In some embodiments, the SBS is due to a congenital deficiency.

In some embodiments, the subject's SBS results from surgical resection of the intestine. In specific embodiments, the subject had a small bowel resection that leaves less than 30% of the small bowel or normal intestine length, less than 25% of the small bowel or normal intestine length, less than 20% of the small bowel or normal intestine length, or less than 15% of the small bowel or normal intestine length, and, in certain embodiments, has at least 2%, at least 3%, at least 5% or at least 10% of the normal intestine length remaining. In certain embodiments, a portion of the large intestine or colon is also resected. In certain embodiments, the resection removes all or a portion of the duodenum, the jejunum or the ileum, or a combination of all or a portion of the duodenum and the jejunum, a combination of all or a portion of the jejunum and the ileum, or a combination of all or a portion of the duodenum, all of the jejunum, and all or a portion of the ileum.

The subject being treated may have SBS Type 1 associated with terminal jejunostomy, Type 2 associated with jejunocolic anastomosis, or Type 3 associated with jejunoileotransversostomy SBS.

In certain embodiments, the subject to be treated for SBS or associated diarrhea is selected from a newborn (0 to 3 months), an infant of about 3 months to 2 years of age, a toddler from about 2 years to about 6 years of age, a child from about 6 years to about 11 years of age, an adolescent from about 12 years to about 18 years of age, and an adult older than about 18 years of age. In embodiments, the subject to be treated is a newborn or an infant that is less than one year of age, and had a small intestine resection as a result of treatment for necrotizing enterocolitis, intestinal anomalies, or midgut volvulus. In one embodiment, the subject to be treated is selected from a newborn (0 to 3 months), an infant of about 3 months to 2 years of age, a toddler from about 2 years to about 6 years of age, a child from about 6 years to about 11 years of age, an adolescent from about 12 years to about 18 years of age, and an adult older than about 18 years of age, and had a small intestine resection as a result of treatment for Crohn's disease, vascular disease, malignancy, radiation enteritis, trauma, or adhesive obstruction. In one embodiment, the subject to be treated is selected from a newborn (0 to 3 months), an infant of about 3 months to 2 years of age, a toddler from about 2 years to about 6 years of age, a child from about 6 years to about 11 years of age, an adolescent from about 12 years to about 18 years of age, and an adult older than about 18 years of age, and had a cholecystectomy.

To treat SBS, crofelemer can be administered, for example, once a day, twice a day, three times a day, or four times or more often as necessary per day. The administered daily dose of crofelemer per the subject's weight is from about 1 mg/kg to about 300 mg/kg per day, specifically from about 3 mg/kg to about 45 mg/kg, about 3 mg/kg to about 10 mg/kg, about 6 mg/kg to about 36 mg/kg per day or about 9 mg/kg to about 30 mg/kg per day or about 15 mg/kg to about 45 mg/kg. A single dose can be administered 3 times a day, or, in embodiments, 2 times per day, 4 times per day or 5 times per day or wherein the single dose is administered with each intake of enteral nutrition, either before, after or during. Thus, the daily dose may be divided into equal doses to be administered throughout the day, for example, 3 equal doses for administration 3 times per day. In embodiments, the daily dose ranges from about 3 mg/kg to about 30 mg/kg or about 9 mg/kg to 30 mg/kg and is divided for administration 3 times per day.

For pediatric subjects, the administered daily dose per the subject's weight is from about 0.5 mg/kg to about 300 mg/kg per day, specifically from about 1 mg/kg to about 200 mg/kg or about 3 mg/kg to about 150 mg/kg per day or about 6 mg/kg to about 100 mg/kg per day. A single pediatric dose can be administered three 3 times a day, or, in embodiments, 2 times per day, 4 times per day or 5 times per day or wherein the single dose is administered with each intake of enteral nutrition, either before, after or during. In an embodiment, the pharmaceutical composition is administered in combination with enteral nutrition, such as by mixing before administration. Accordingly, the daily dose may be divided into equal doses to be administered throughout the day, for example, 3 equal doses for administration 3 times per day. In embodiments for pediatric subjects, the daily dose ranges from about 1 mg/kg to about 100 mg/kg or about 3 mg/kg to about 30 mg/kg or about 3 mg/kg or about 9 mg/kg or about 30 mg/kg that is divided for administration three times a day.

As mentioned above, the pharmaceutical composition can be administered to the subject in conjunction with enteral nutrition. The pharmaceutical composition may be administered before, during, or after the administration of enteral nutrition.

For those subjects with SBS receiving parenteral nutrition (PN), the pharmaceutical composition comprising crofelemer as described herein can be administered in combination. In embodiments, the subject is administered PN immediately after surgical resection to meet the nutritional needs of the subject along with the pharmaceutical composition to address diarrhea associated with SBS. Parenteral nutrition may be administered for the first 7 to 10 days, or even longer, such as 15, 20 or 30 days, after surgical resection, including cases in which the subject receives all nutrition or a portion of the subject's nutritional needs through PN. As the intestine adapts, the need for PN may be reduced and the subject may, in certain circumstances, be weaned off the need for PN. In specific embodiments, the subject may be administered the pharmaceutical composition during the 2, 3, 4, 5, 6, 7, 8, 9, 10, 12, or 15 week post-operative period during which PN is administered and/or in the PN weaning phase and/or in the 2, 3, 4, 5, 6, 7, 8, 9, or 10 week period following the PN weaning phase. The pharmaceutical composition may be administered in conjunction with PN chronically or even after the PN is reduced or stopped altogether as the subject adapts to a shorter small intestine.

In specific embodiments, the subject needs long term or life time PN and is also administered the pharmaceutical composition comprising crofelemer as described herein long term, chronically or for life. Long term PN can result in serious metabolic complications, including hepatic and biliary disorders, such as, steatosis, fibrosis, and cholestasis, which can progress to fulminant liver failure. Accordingly, methods are provided for treating secretory diarrhea in subjects suffering from SBS who have a hepatic and/or biliary disorder. In embodiments, the administration of the pharmaceutical composition as described herein at a therapeutically effective amount reduces the severity or impact of the hepatic or biliary disorder, including steatosis, fibrosis or cholestasis, and reduces the risk or incidence of liver failure.

Administration of the pharmaceutical composition described herein at a therapeutically effective amount reduces the need for PN by at least 15%, at least 20%, at least 25%, or at least 30% over the course of about 12, 15, 20, 24, 28, or 32 weeks of administration of the pharmaceutical composition. In particular embodiments, the subject's parenteral nutrition daily amount can be decreased based on when the subject's degree of edema has not increased. In a further embodiment, the subject on PN can increase the daily amount of enteral nutrition over the course of about 12, 15, 20, 24, 28, or 32 weeks of administration of the pharmaceutical composition.

In embodiments, the pharmaceutical composition is administered until symptoms of SBS or diarrhea are ameliorated and then pharmaceutical composition is discontinued. However, the pharmaceutical composition is suitable for long term, continuous use to ameliorate symptoms.

### VI. Methods of Treating MVID or TE and Diarrhea Associated Therewith

Congenital Diarrheal Disorders are a heterogeneous group of diarrheal disorders, primarily hereditary, that present early in life, typically in infancy, with severe watery diarrhea, imbalanced serum chemistry and failure to thrive. As these disorders rapidly intensify, immediate and long-term total parenteral nutrition (TPN) becomes pertinent and may be the only treatment option in most cases of CDDs. Infants are often hospitalized to receive supportive treatment, nutritional rehabilitation, and drugs. Eventually, bowel transplantation becomes required for survival. Hematopoietic stem cell transplantation (HSCT) is used in conditions with an underlying immune defect, similar to bowel transplantation; the invasive treatments carry a significant risk for complication and mortality. In all aspects, CDDs are life-threatening conditions with high chance of mortality of life-long morbidity.

Microvillus Inclusion Disease (MVID) is one type of CDD. It is caused by a mutation in the MYO5B gene which results in a decrease of absence of myosin Vb function. In cells that line the small intestine, a lack of myosin Vb function changes the cell polarity, which prevents the proper production of structures known as microvilli. Microvilli ordinarily help absorb nutrients from food as it passes through the intestine. Epithelial cells with poorly formed microvilli reduce the intestine's ability to absorb nutrients. There are no anti-diarrheal drugs approved for the treatment of diarrhea associated with MVID.

Tufting Enteropathy (TE) is another type of CDD. TE is typically diagnosed by its characteristic histological features, including villous atrophy, crypt hyperplasia and focal epithelial tufts consisting of densely packed enterocytes. Mutations in the EPCAM and SPINT2 genes have been identified as the etiology for this disease. The pathogenesis involves intricate overlapping mechanisms that include defects in epithelial cells and enterocyte function ranging from enterocyte disorganization, impaired enzymes and metabolism, defective epithelial trafficking/polarity, and altered cell differentiation. There are no anti-diarrheal drugs approved for the treatment of diarrhea associated with TE.

Crofelemer is an antagonist of cystic fibrosis transmembrane conductance regulator (CFTR) and calcium-activated chloride channels (CaCCs) that mediate intestinal fluid secretion by the enterocytes. By inhibiting these channels, crofelemer may treat or ameliorate diarrheal symptoms associated with MVID or TE. In addition, crofelemer has significant treatment potential for such diseases, particularly in infants, due to minimal absorption of crofelemer and, therefore, high safety profile.

Treating SBS or associated symptoms with a therapeutically effective amount of the pharmaceutical composition as described herein can result in an improvement the consistency and frequency of stool or in the sensations associated therewith. In embodiments, treating SBS or associated symptoms with the pharmaceutical composition will decrease the number of bowel movements per day (frequency of stools), decrease the number of watery bowel movements per day (frequency of abnormal stools), decrease the urgency or fecal incontinence, decrease the symptom severity (abdominal pain or discomfort), or decrease the daily stool consistency score (watery to formed). This decrease may be measured from a baseline. The baseline may be determined in the days prior to treatment with the pharmaceutical composition.

The methods disclosed herein involved the administration of therapeutically effective amounts of a pharmaceutical composition comprising lyophilized crofelemer or crofelemer is powder form as described herein to subjects having MVID or TE.

### MVID

A method in accordance with the present disclosure involves treating MVID or diarrhea associated with MVID in a subject by administering to the subject in need thereof a therapeutically effective amount of the pharmaceutical compositions disclosed herein. The subject is preferably a human. The pharmaceutical composition may be a reconstituted liquid formulation, including, in an embodiment, at a concentration of about 2 mg/mL to about 120 mg/mL, reconstituted from a powder or dispersible form of crofelemer, including, in an embodiment, a lyophilized form of crofelemer.

The subject to be treated can be of any age, such as a newborn (0 to 3 months), an infant of about 3 months to 2 years of age, a toddler from about 2 years to about 6 years of age, a child from about 6 years to about 11 years of age, an adolescent from about 12 years to about 18 years of age, and an adult older than about 18 years of age. In embodiments, the subject to be treated is a newborn, an infant or a toddler (between 2 and 6 years old).

To treat MVID, the pharmaceutical composition as described herein can be administered, for example, once a day, twice a day, three times a day, or four times or more often as necessary per day. The administered daily dose of crofelemer per the subject's weight is from about 1 mg/kg to about 300 mg/kg per day, specifically from about 3 mg/kg to about 45 mg/kg, about 3 mg/kg to about 10 mg/kg, about 6 mg/kg to about 36 mg/kg per day or about 9 mg/kg to about 30 mg/kg per day or about 15 mg/kg to about 45 mg/kg. A single dose can be administered 3 times a day, or, in embodiments, 2 times per day, 4 times per day or 5 times per day or wherein the single dose is administered with each intake of enteral nutrition, either before, after or during. Thus, the daily dose may be divided into equal doses to be administered throughout the day, for example, 3 equal doses for administration 3 times per day. In embodiments, the daily dose ranges from about 3 mg/kg to about 30 mg/kg or about 9 mg/kg to 30 mg/kg and is divided for administration 3 times per day.

As mentioned above, the pharmaceutical composition is can be administered to the subject in conjunction with enteral nutrition. The pharmaceutical composition may be administered before, during, or after the administration of enteral nutrition.

Subjects with MVID typically receive parenteral nutrition (PN), and the pharmaceutical composition comprising crofelemer as described herein can be administered in combination. The subject are administered PN soon after being born to meet nutritional needs. Parenteral nutrition is often lifelong unless a short bowel transplant is obtained. Subjects may be weaned from PN after a short bowel transplant. In specific embodiments, the subject may be administered the pharmaceutical composition for a continuous period prior to receiving a short bowel transplant. In addition or alternatively, the subject may be administered the pharmaceutical composition during the 2, 3, 4, 5, 6, 7, 8, 9, or 10 weeks following the post-transplant period during which PN is administered and/or in the PN weaning phase and/or in the 2, 3, 4, 5, 6, 7, 8, 9, or 10 weeks following the PN weaning phase. The pharmaceutical composition may be administered in conjunction with PN chronically or even after the PN is reduced or stopped altogether as the subject adapts to the transplanted short bowel.

In specific embodiments, the subject needs long term or life time PN and is also administered the pharmaceutical composition comprising crofelemer as described herein long term, chronically or for life. Long term PN can result in serious metabolic complications, including hepatic and biliary disorders, such as, steatosis, fibrosis, and cholestasis, which can progress to fulminant liver failure. Accordingly, methods are provided for treating secretory diarrhea in subjects suffering from MVID who have a hepatic and/or biliary disorder. In embodiments, the administration of the pharmaceutical composition as described herein at a therapeutically effective amount reduces the severity or impact of the hepatic or biliary disorder, including steatosis, fibrosis or cholestasis, and reduces the risk or incidence of liver failure.

Administration of the pharmaceutical composition described herein at a therapeutically effective amount to treat MVID reduces the need for PN by at least 15%, at least 20%, at least 25%, or at least 30% over the course of about 12, 15, 20, 24, 28, or 32 weeks of administration of the pharmaceutical composition. In particular embodiments, the subject's PN daily amount can be decreased based on when the subject's degree of edema has not increased. In a further embodiment, the subject on PN can increase the daily amount of enteral nutrition over the course of about 12, 15, 20, 24, 28, or 32 weeks of administration of the pharmaceutical composition.

Treating MVID or associated symptoms with a therapeutically effective amount of the pharmaceutical composition as described herein can result in an improvement the consistency and frequency of stool or in the sensations associated therewith. In embodiments, treating MVID or associated symptoms with the pharmaceutical composition will decrease the number of bowel movements per day (frequency of stools), decrease the number of watery bowel movements per day (frequency of abnormal stools), decrease the urgency or fecal incontinence, decrease the symptom severity (abdominal pain or discomfort), or decrease the daily stool consistency score (watery to formed). This decrease may be measured from a baseline. The baseline may be determined in the days prior to treatment with the pharmaceutical composition.

The pharmaceutical compositions comprising crofelemer as described herein reduces or ameliorates the MVID symptoms, diarrhea associated MVID symptoms, or diarrhea associated with MVID in a subject.

### Tufted Enteropathy

A method in accordance with the present disclosure involves treating TE or diarrhea associated with TE in a subject by administering to the subject in need thereof a therapeutically effective amount of the pharmaceutical compositions disclosed herein. The subject is preferably a human. The pharmaceutical composition may be a reconstituted liquid formulation, including, in an embodiment, at a concentration of about 2 mg/mL to about 120 mg/mL, reconstituted from a powder or dispersible form of crofelemer, including, in an embodiment, a lyophilized form of crofelemer.

The subject to be treated for TE can be of any age, such as a newborn (0 to 3 months), an infant of about 3 months to 2 years of age, a toddler from about 2 years to about 6 years of age, a child from about 6 years to about 11 years of age, an adolescent from about 12 years to about 18 years of age, and an adult older than about 18 years of age. In embodiments, the subject to be treated is a newborn, an infant or a toddler (between 2 and 6 years old).

To treat TE, the pharmaceutical composition described herein can be administered, for example, once a day, twice a day, three times a day, or four times or more often as necessary per day. The administered daily dose of crofelemer per the subject's weight is from about 1 mg/kg to about 300 mg/kg per day, specifically from about 3 mg/kg to about 45 mg/kg, about 3 mg/kg to about 10 mg/kg, about 6 mg/kg to about 36 mg/kg per day or about 9 mg/kg to about 30 mg/kg per day or about 15 mg/kg to about 45 mg/kg. A single dose can be administered 3 times a day, or, in embodiments, 2 times per day, 4 times per day or 5 times per day or wherein the single dose is administered with each intake of enteral nutrition, either before, after or during. Thus, the daily dose may be divided into equal doses to be administered throughout the day, for example, 3 equal doses for administration 3 times per day. In embodiments, the daily dose ranges from about 3 mg/kg to about 30 mg/kg or about 9 mg/kg to 30 mg/kg and is divided for administration 3 times per day.

As mentioned above, the pharmaceutical composition is can be administered to the subject in conjunction with enteral nutrition. The pharmaceutical composition may be administered before, during, or after the administration of enteral nutrition.

Subjects with TE typically receive parenteral nutrition (PN), and the pharmaceutical composition comprising crofelemer as described herein can be administered in combination. The subject are administered PN soon after being born to meet nutritional needs. Parenteral nutrition is often lifelong though a few subjects do naturally improves over time and others may successfully obtain a short bowel transplant. Subjects may be weaned from PN after a short bowel transplant or an improvement in bowel function. In specific embodiments, the subject may be administered the pharmaceutical composition for a continuous period prior to receiving a short bowel transplant. In addition or alternatively, the subject may be administered the pharmaceutical composition during the 2, 3, 4, 5, 6, 7, 8, 9, or 10 weeks following the post-transplant period during which PN is administered and/or in the PN weaning phase and/or in the 2, 3, 4, 5, 6, 7, 8, 9, or 10 weeks following the PN weaning phase. The pharmaceutical composition may be administered in conjunction with PN chronically or even after the PN is reduced or stopped altogether as the subject adapts to the transplanted short bowel.

In specific embodiments, the subject needs long term or life time PN and is also administered the pharmaceutical composition comprising crofelemer as described herein long term, chronically or for life. Long term PN can result in serious metabolic complications, including hepatic and biliary disorders, such as, steatosis, fibrosis, and cholestasis, which can progress to fulminant liver failure. Accordingly, methods are provided for treating secretory diarrhea in subjects suffering from TE who have a hepatic and/or biliary disorder. In embodiments, the administration of the pharmaceutical composition as described herein at a therapeutically effective amount reduces the severity or impact of the hepatic or biliary disorder, including steatosis, fibrosis or cholestasis, and reduces the risk or incidence of liver failure.

Administration of the pharmaceutical composition described herein at a therapeutically effective amount to treat TE reduces the need for PN by at least 15%, at least 20%, at least 25%, or at least 30% over the course of about 12, 15, 20, 24, 28, 32, 36, or 40 weeks of administration of the pharmaceutical composition. In particular embodiments, the subject's PN daily amount can be decreased based on when the subject's degree of edema has not increased. In a further embodiment, the subject on PN can increase the daily amount of enteral nutrition over the course of about 12, 15, 20, 24, 28, 32, 36, or 40 weeks of administration of the pharmaceutical composition.

Treating TE or associated symptoms with a therapeutically effective amount of the pharmaceutical composition as described herein can result in an improvement the consistency and frequency of stool or in the sensations associated therewith. In embodiments, treating TE or associated symptoms with the pharmaceutical composition will decrease the number of bowel movements per day (frequency of stools), decrease the number of watery bowel movements per day (frequency of abnormal stools), decrease the urgency or fecal incontinence, decrease the symptom severity (abdominal pain or discomfort), or decrease the daily stool consistency score (watery to formed). This decrease may be measured from a baseline. The baseline may be determined in the days prior to treatment with the pharmaceutical composition.

The pharmaceutical compositions comprising crofelemer as described herein reduces or ameliorates the TE symptoms, diarrhea associated TE symptoms, or diarrhea associated with TE in a subject.

### VII. Kits

Kits are also provided herein, for example, kits for treating a disorder associated with SBS, diarrhea associated with SBS, MVID, TE, or diarrhea associated with MVID or TE. The kits may contain, for example, crofelemer or a pharmaceutical composition comprising crofelemer and instructions for use. The instructions for use may contain prescribing information, dosage information, storage information, and the like.

Label instructions include, for example, instructions for administering the composition in connection with treating a disorder associated with short bowel syndrome (SBS) or diarrhea associated therewith SBS, MVID, TE, or diarrhea associated with MVID or TE, and instructions for treating a disorder associated with short bowel syndrome (SBS) or diarrhea associated therewith SBS, MVID, TE, or diarrhea associated with MVID or TE.

In certain embodiments, the disorder is associated with Short Bowel Syndrome (SBS).

In particular embodiments, the disorder is associated with Microvillus Inclusion Disease (MVID).

In particular embodiments, the disorder is associated with Microvillus Inclusion Disease (TE).

In particular embodiments, the kits further comprise from about 15 to about 20 individual vials of the pharmaceutical composition in lyophilized or powder form.

### VIII. Examples

### A. Example 1—Lyophilization of Crofelemer Compositions

The objective of the study is produce a lyophilized crofelemer composition. The target is to process 3 crofelemer bulk solutions and 3 crofelemer placebo solutions. Two target active presentations of 1.5 g and 450 mg were processed as well as an active sublot with reduced solids to mimic the solids content of the crofelemer placebo where Benzoic Acid loss was observed at a higher magnitude than the active solutions, presumably due to a less dense cake. The Placebo formulations consisted of different preservative combinations at varying levels of Sodium Benzoate and one with Potassium Sorbate included. It was found that a shift to a higher chamber pressure (ex. 600 microns) at the onset of secondary drying during the lyophilization process minimized benzoic acid sublimation and loss. Each candidate was stoppered at approximately 30 hours, 40 hours and 50 hours into the drying segment. Approximately 25 samples of each sublot were processed.

The formulations of each sample are shown in Table 1 below.

TABLE 1

| | | Sodium | | | | | FD&C |
|---|---|---|---|---|---|---|---|
| Rx | Crofelemer | Benzoate | Sucralose | Mannitol | FD&C | FD&C | Yellow |
| ID | (mg/mL) | (mg/mL) | (mg/L) | (mg/mL) | Blue #2 | Red #40 | #5 |
| A9 | 150 | 2.25 | 18.75 | — | — | — | — |
| A10 | 45 | 2.25 | 5.62 | — | — | — | — |
| A11 | 35 | 2.25 | 4.36 | — | — | — | — |
| P12 | — | 2.25 | 1.5 | 30 | 0.18 | 2.39 | 1.07 |
| P13 | — | 2.25 | 1.5 | 30 | 0.18 | 2.39 | 1.07 |
| P14 | — | 4.50 | 1.5 | 30 | 0.18 | 2.39 | 1.07 |

| | Quinine Sulfate | Citric Acid | Sodium Citrate | Potassium |
|---|---|---|---|---|
| Rx | (Dihydrate) | (anhydrous) | (Dihydrate) | Sorbate |
| ID | (mg/mL) | (mg/mL) | (mg/mL) | (mg/mL) |
| A9 | — | 2.05 | 1.28 | — |
| A10 | — | 2.05 | 1.28 | — |
| A11 | — | 0.48 | 0.30 | — |
| P12 | 0.3 | 2.05 | 1.28 | — |
| P13 | 0.3 | 2.05 | 1.28 | 3.0 |
| P14 | 0.3 | 2.05 | 1.28 | — |

20 mm reaction vials were prepared according to the crofelemer and excipient concentrations shown in Table 1 and dissolved in purified water, USP. The vials were stored at room temperature until lyophilization. The lyophilizer cycle program was prepared according to the parameters in Table 2.

TABLE 2

| | Shelf Temp. | Soak Time | Ramping Rate | Chamber | |
|---|---|---|---|---|---|
| Step | Setpoint (° C.) | (hours) | (° C./hour) | Pressure | |
| Load | 5 | 2 | — | ≤12 | PSIA |
| Freezing, Step 1 | — | — | 30 | | |
| | −40 | 3 | — | | |
| Annealing | — | — | 30 | | |
| | −15 | 6 | — | | |
| Freezing, Step 2 | — | — | 30 | | |
| | −40 | 4 | — | | |
| Primary Drying | — | — | 30 | 60 | microns |
| | 25 | ~30 | — | | |
| Secondary | — | — | — | 600 | microns |
| Drying | 25 | ~20 | | | |
| Stoppering | 25 | — | — | 8.0 ± 0.3 | PSIA |

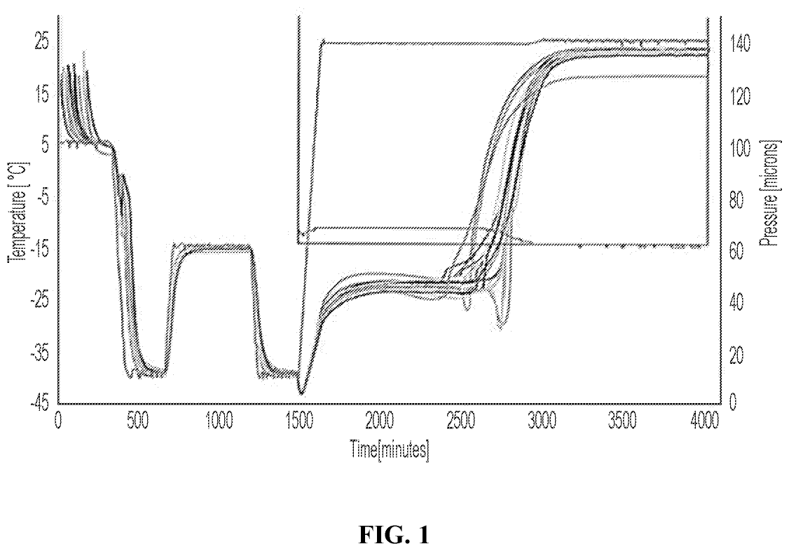

A data plot showing representative Temperature and Pressure as a function of time for the lyophilization runs is shown in FIG. 1. The representative data shown is for 1.5 g vials.

### B. Example 2—Sweetener Studies

Candidates for high intensity sweetener excipients were analyzed for their applicability for the present pharmaceutical composition. Potential candidates should provide adequate sweetness within the acceptable daily intake limits and should minimize the potential for metabolic effects in subjects. The findings are presented in Table 3 below. A usage of 10-15 mg/mL sucralose for the 100 mg/mL crofelemer solution was found to overcome the intensity of bitterness of crofelemer.

TABLE 3

| | | Us Acceptable Daily | |
|---|---|---|---|
| | Sweetness Intensity | Intake, mg/Body | |
| Sweetener | Reference to Sucrose | Weight | Comments |
| Acesulfame | 200x | 15 | Relative Low |
| Potassium (Ace-K) | | | intensity |
| Advantame | 20,000x | 32.8 | Potential Candidate |
| Aspartame | 200x | 50 | Relative Low |
| | | | Intensity |
| Neotame | 7,000-13,000x | 0.3 | Low Acceptable Limit |
| Saccharin | 200-700x | 15 | Potential Candidate |
| Sucralose | 600x | 5 | Potential Candidate |

### C. Example 3—Stability Studies

A study was performed to evaluate solution stability of Crofelemer in water at 3 concentrations: 10, 25 and 50 mg/mL. The results are shown in Table 4. Each solution was adjusted to a final solution pH of pH 4 (using 1% hydrochloric acid w/w) and pH 6 (using sodium hydroxide 10% w/w). Samples representing each of the six different solutions were stored at 2° C. to 8° C. (stated as 3 C in the table) and ambient room temperature (stated as RT in the table). All samples were examined for appearance on a weekly basis. The appearance specification is ‘clear liquid solution’. Yes indicates that the samples met the appearance spec. No indicates that the solution did not meet the appearance specification.

TABLE 4

| Strength | | Test | Day | | | | | | | | | | | |
|---|---|---|---|---|---|---|---|---|---|---|---|---|---|---|
| (mg/mL) | pH | Condition | 7 | 14 | 21 | 28 | 35 | 42 | 49 | 57 | 64 | 71 | 78 | 85 |
| 10 | 4.3 | 3 C. | Yes | Yes | Yes | Yes | Yes | Yes | Yes | Yes | Yes | Yes | Yes | No |
| | | RT | Yes | Yes | Yes | Yes | Yes | Yes | Yes | No | No | No | No | No |
| | 6.1 | 3 C. | Yes | Yes | Yes | Yes | Yes | Yes | Yes | Yes | Yes | Yes | Yes | No |
| | | RT | Yes | Yes | Yes | Yes | Yes | Yes | Yes | Yes | Yes | Yes | Yes | No |
| 25 | 4.2 | 3 C. | Yes | Yes | No | No | No | No | No | No | No | No | No | No |
| | | RT | Yes | Yes | Yes | Yes | No | No | No | No | No | No | No | No |
| | 6.1 | 3 C. | Yes | Yes | Yes | Yes | Yes | Yes | Yes | Yes | Yes | Yes | Yes | No |
| | | RT | Yes | Yes | Yes | Yes | Yes | Yes | Yes | Yes | Yes | Yes | Yes | No |
| 50 | 4.1 | 3 C. | Yes | Yes | No | No | No | No | No | No | No | No | No | No |
| | | RT | Yes | Yes | Yes | Yes | No | No | No | No | No | No | No | No |
| | 6.2 | 3 C. | Yes | Yes | Yes | Yes | Yes | Yes | Yes | Yes | Yes | Yes | No | No |
| | | RT | Yes | Yes | Yes | Yes | Yes | Yes | Yes | Yes | Yes | Yes | No | No |

### D. Example 4—Formulations for Clinical Trials

The drug product (DP) is crofelemer powder for oral solution at 1.5 g and 450 mg of crofelemer. The DP is a lyophilized powder of the active pharmaceutical ingredient, crofelemer, packaged in a 30 mL type 1 clear glass 20 mm opening vial with a grey butyl B2-TR igloo style stopper and a 20 mm Flip-Off, TruEdge® seal.

Prior to administration, DP at 1.5 g and 450 mg of crofelemer is reconstituted with 14 mL of water to a reconstituted volume of approximately 15 mL. The composition of the DP is presented in Table 5.

TABLE 5

| | Amount per Vial | | |
|---|---|---|---|
| Ingredient | 1.5 g Vial | 450 mg Vial | Function |
| Crofelemer ^{a} | 1500 mg | 450 mg | Active Ingredient |
| Mannitol | — | 300 mg | Bulking agent |
| Sucralose | 187.5 mg | 56.2 mg | Sweetener |
| Sodium Benzoate | 22.5 mg | 22.5 mg | Preservative |
| Citric Acid monohydrate | 22.4 mg | 23.1 mg | pH adjusting agent |
| Sodium Citrate dihydrate | 12.8 mg | 13.5 mg | pH adjusting agent |
| Citric Acid solution, 1M ^{b} | ^{c} | ^{c} | pH adjusting agent |
| Sodium Citrate dihydrate | ^{c} | ^{c} | pH adjusting agent |
| solution, 1M ^{b} | | | |
| Purified Water ^{d} | qs to 10 mL | qs to 10 mL | Processing fluid |

^{a} Corrected for potency
^{b} Prepared from Multi-Compendial (MC) reagent and Purified Water (USP).
^{c} Used for pH adjustment of the fill solutions to 4.1 +/− 0.2 pH units prior to vial filling and lyophilization
^{d} Removed during processing

### E. Example 5—Manufacturing Process

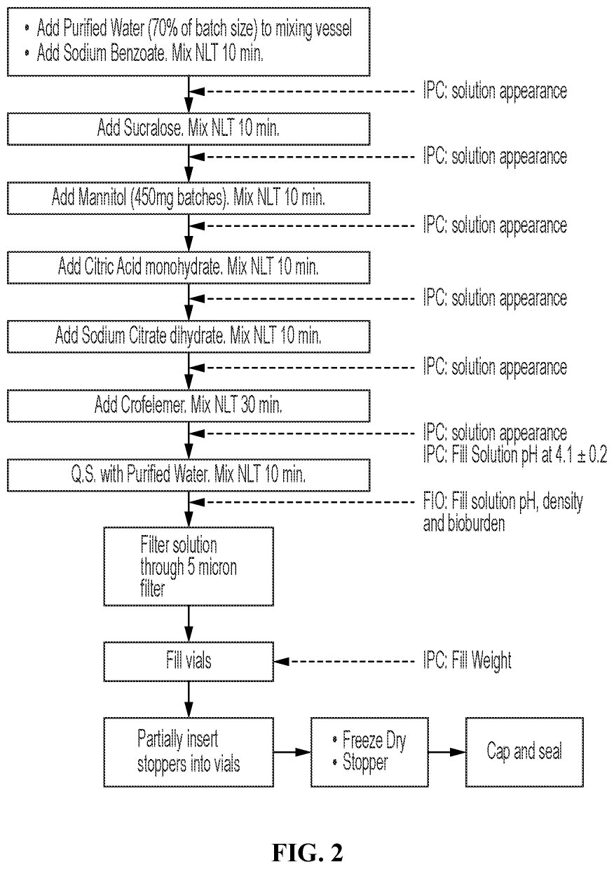

DP at 1.5 g and 450 mg of crofelemer are manufactured by a standard lyophilization process as shown in FIG. 2. Excipients and the drug substance are dissolved in purified water and passed through a 5-micron filter for removing particulates prior to vial filling. The partially stoppered vials are placed in a freeze dryer which removes the water via sublimation. The stoppers are fully inserted into the vials in the dryer. The vials are removed and then capped and sealed.

The same process and equipment are used for manufacturing the DP at 1.5 g of crofelemer and DP at 450 mg of crofelemer. The lyophilization cycle is described in Table 6.

TABLE 6

| Freeze Drying Step | Parameter | Set-Point | | Soak Time | |
|---|---|---|---|---|---|
| Initial freezing | Shelf temperature | −40° | C. | 240 | min. |
| Annealing | Shelf temperature | −15° | C. | 480 | min. |
| Freezing | Shelf temperature | −40° | C. | 240 | min. |
| Primary/secondary drying | Shelf temperature | 25° | C. | 2400 | min. |

| | | | |
|---|---|---|---|
| Primary/secondary drying | Chamber pressure | 60 | microns-Hg* |

*0.000079 atmospheres

### F. Example 6—Stability of Drug Product at 1.5 g at Long Term Storage Conditions

Table 7 shows the results of 6 month stability studies performed at a room temperature of 25° C.±2° C. and relative humidity of 60%±5% for DP at 1.5 g of crofelemer.

TABLE 7

| | | Test | Time | | | | |
|---|---|---|---|---|---|---|---|
| Quality Attribute | | Specification | 0 months | | 1 month | 3 months | 6 months |
| Appearance, | | Reddish | Conforms | | Conforms | Conforms | Conforms |
| Lyophilized | | brown to | | | | | |
| | | greenish | | | | | |
| | | brown | | | | | |
| | | powder | | | | | |
| Appearance, | | Dark Red | Conforms | | Conforms | Conforms | Conforms |
| Reconstituted | | solution. No | | | | | |
| Solution | | visible | | | | | |
| | | insoluble | | | | | |
| | | matter | | | | | |
| pH | | 3.5-5.0 | 4.2 | | 4.2 | 4.2 | 4.4 |
| Identified | Gallocatechin | | ND | | ND | ND | ND |
| Impurities | Epigallocatechin | | ND | | <LOQ | <LOQ | <LOQ |
| | Catechin | | ND | | ND | ND | ND |
| | Epicatechin | | ND | | <LOQ | ND | ND |
| | Procyanidin B1 | | 0.1% | | <LOQ | ND | 0.1% |
| | Procyanidin B2 | | 0.1% | | ND | ND | 0.1% |
| Individial | | | 0.1% | | <LOQ | ND | 0.1% |
| Unknown | | | | | | | |
| Total Related | | NMT 3.0%, | 0.1% | | ND | ND | 0.1% |
| Substances | | Area | | | | | |
| Sodium | | 1.0-2.0 | 1.44 | | N/A | N/A | N/A |
| Benzoate | | mg/mL | | | | | |
| Water | | NMT 10%, | 1.16% | | 0.76% | 0.97% | 0.74% |
| Content | | w/w | | | | | |
| Reconstitution | | NMT 15 min | ≤3 | min | ≤3 min | ≤3 min | ≤2 min |
| Time | | | | | | | |
| Microbial | Total Aerobic | NMT 200 | <2 | cfu/g | N/A | N/A | N/A |
| Limits | Bacterial Count | CFU/mL | | | | | |
| | Total Combined | NMT 20 | <2 | cfu/g | N/A | N/A | N/A |

| | | | | | |
|---|---|---|---|---|---|
| Molds and Yeast | CFU/mL | | | | |
| E. Coli | Absent | Absent | N/A | N/A | N/A |
| | (negative) | | | | |

### G. Example 7—Stability of Drug Product at 1.5 g at Accelerated Storage Conditions

Table 8 shows the results of 6 month stability studies performed at accelerated storage conditions of 40° C.±2° C. and relative humidity of 75%±5% for DP at 1.5 g of crofelemer.

TABLE 8

| | | Test | Time | | | | |
|---|---|---|---|---|---|---|---|
| Quality Attribute | | Specification | 0 months | | 1 month | 3 months | 6 months |
| Appearance, | | Reddish | Conforms | | Conforms | Conforms | Conforms |
| Lyophilized | | brown to | | | | | |
| | | greenish | | | | | |
| | | brown | | | | | |
| | | powder | | | | | |
| Appearance, | | Dark Red | Conforms | | Conforms | Conforms | Conforms |
| Reconstituted | | solution. No | | | | | |
| Solution | | visible | | | | | |
| | | insoluble | | | | | |
| | | matter | | | | | |
| pH | | 3.5-5.0 | 4.2 | | 4.2 | 4.2 | 4.4 |
| Identified | Gallocatechin | | ND | | ND | ND | ND |
| Impurities | Epigallocate chin | | ND | | <LOQ | <LOQ | <LOQ |
| | Catechin | | ND | | ND | ND | ND |
| | Epicatechin | | ND | | <LOQ | ND | ND |
| | Procyanidin BI | | 0.1% | | ND | ND | ND |
| | Procyanidin B2 | | ND | | <LOQ | <LOQ | ND |
| Individial | | | 0.1% | | <LOQ | <LOQ | <LOQ |
| Unknown | | | | | | | |
| Total Related | | NMT 3.0%, | 0.1% | | ND | ND | 0.1% |
| Substances | | Area | | | | | |
| Water | | NMT 10%, | 1.2% | | 0.7% | 0.85% | 0.74% |
| Content | | w/w | | | | | |
| Reconstitution | | NMT 15 min | ≤3 | min | ≤3 min | ≤3 min | ≤3 min |
| Time | | | | | | | |
| Microbial | Total Aerobic | NMT 200 | <2 | cfu/g | N/A | N/A | N/A |
| Limits | Bacterial Count | CFU/mL | | | | | |
| | Total Combined | NMT 20 | <2 | cfu/g | N/A | N/A | N/A |

| | | | | | |
|---|---|---|---|---|---|
| Molds and Yeast | CFU/mL | | | | |
| E. Coli | Absent | Absent | N/A | N/A | N/A |
| | (negative) | | | | |

### H. Example 8—Stability of Drug Product at 450 mg at Long Term Storage Conditions

Table 9 shows the results of 6 month stability studies performed at a room temperature of 25° C.±2° C. and relative humidity of 60%±5% for DP at 450 mg of crofelemer.

TABLE 9

| | | Test | Time | | | | |
|---|---|---|---|---|---|---|---|
| Quality Attribute | | Specification | 0 months | | 1 month | 3 months | 6 months |
| Appearance, | | Reddish | Conforms | | Conforms | Conforms | Conforms |
| Lyophilized | | brown to | | | | | |
| | | greenish | | | | | |
| | | brown | | | | | |
| | | powder | | | | | |
| Appearance, | | Dark Red | Conforms | | Conforms | Conforms | Conforms |
| Reconstituted | | solution. No | | | | | |
| Solution | | visible | | | | | |
| | | insoluble | | | | | |
| | | matter | | | | | |
| pH | | 3.5-5.0 | 4.3 | | 4.3 | 4.3 | 4.5 |
| Identified | Gallocatechin | | ND | | ND | ND | ND |
| Impurities | Epigallocatechin | | ND | | ND | ND | <LOQ |
| | Catechin | | ND | | ND | ND | ND |
| | Epicatechin | | <LOQ | | ND | ND | ND |
| | Procyanidin B1 | | ND | | <LOQ | <LOQ | ND |
| | Procyanidin B2 | | ND | | ND | ND | ND |
| Individial | | | 0.1% | | ND | <LOQ | 0.1% |
| Unknown | | | | | | | |
| Total Related | | NMT 3.0%, | 0.1% | | ND | ND | 0.1% |
| Substances | | Area | | | | | |
| Water | | NMT 10%, | 2.84% | | 2.75% | 2.74% | 3.36% |
| Content | | w/w | | | | | |
| Reconstitution | | NMT 15 min | ≤2 | min | ≤1 min | ≤1 min | ≤2 min |
| Time | | | | | | | |
| Microbial | Total Aerobic | NMT 200 | <1 | cfu/g | N/A | N/A | N/A |
| Limits | Bacterial Count | CFU/mL | | | | | |
| | Total Combined | NMT 20 | <1 | cfu/g | N/A | N/A | N/A |

| | | | | | |
|---|---|---|---|---|---|
| Molds and Yeast | CFU/mL | | | | |
| E. Coli | Absent | Absent | N/A | N/A | N/A |
| | (negative) | | | | |

### I. Example 9—Stability of Drug Product at 450 mg at Accelerated Storage Conditions

Table 10 shows the results of 6 month stability studies performed at accelerated storage conditions of 40° C.±2° C. and relative humidity of 75%±5% for DP at 450 mg of crofelemer.

TABLE 10

| | | Test | Time | | | | |
|---|---|---|---|---|---|---|---|
| Quality Attribute | | Specification | 0 months | | 1 month | 3 months | 6 months |
| Appearance, | | Brown | Conforms | | Conforms | Conforms | Conforms |
| Lyophilized | | powder | | | | | |
| Appearance, | | Dark Red | Conforms | | Conforms | Conforms | Conforms |
| Reconstituted | | solution. No | | | | | |
| Solution | | visible | | | | | |
| | | insoluble | | | | | |
| | | matter | | | | | |
| pH | | 3.5-5.0 | 4.3 | | 4.3 | 4.3 | 4.5 |
| Identified | Gallocatechin | | ND | | ND | ND | ND |
| Impurities | Epigallocatechin | | ND | | <LOQ | ND | <LOQ |
| | Catechin | | ND | | ND | ND | ND |
| | Epicatechin | | <LOQ | | ND | ND | ND |
| | Procyanidin B1 | | ND | | <LOQ | <LOQ | ND |
| | Procyanidin B2 | | ND | | ND | ND | ND |
| Individial | | | 0.1% | | ND | <LOQ | 0.1% |
| Unknown | | | | | | | |
| Total Related | | NMT 3.0%, | 0.1% | | ND | ND | 0.1% |
| Substances | | Area | | | | | |
| Water | | NMT 10%, | 2.84% | | 2.74% | 2.76% | 2.74% |
| Content | | w/w | | | | | |
| Reconstitution | | NMT 15 min | ≤2 | min | ≤1 min | ≤1 min | ≤2 min |
| Time | | | | | | | |
| Microbial | Total Aerobic | NMT 200 | <1 | cfu/g | N/A | N/A | N/A |
| Limits | Bacterial Count | CFU/mL | | | | | |
| | Total Combined | NMT 20 | <1 | cfu/g | N/A | N/A | N/A |

| | | | | | |
|---|---|---|---|---|---|
| Molds and Yeast | CFU/mL | | | | |
| E. Coli | Absent | Absent | N/A | N/A | N/A |
| | (negative) | | | | |

### J. Example 10—Stability of Reconstituted Solutions for Drug Product at 1.5 g at 2-8° C.

Table 11 shows the results of 2 week stability studies performed at storage conditions of 2-8° C. for reconstituted DP with 1.5 g of crofelemer.

TABLE 11

| | | Test | Time | | |
|---|---|---|---|---|---|
| Quality Attribute | | Specification | 0 weeks | 1 week | 2 weeks |
| Appearance, | | Dark Red | Conforms | Conforms | Conforms |
| Reconstituted | | solution. No | | | |
| Solution | | visible | | | |
| | | insoluble | | | |
| | | matter | | | |
| pH | | 3.5-5.0 | 4.3 | 4.2 | 3.9 |
| Identified | Gallocatechin | | ND | <LOQ | ND |
| Impurities | Epigallocatechin | | <LOQ | <LOQ | ND |
| | Catechin | | ND | ND | ND |
| | Epicatechin | | ND | ND | ND |
| | Procyanidin B1 | | ND | ND | ND |
| | Procyanidin B2 | | ND | ND | ND |
| Individial | | | 0.1% | 0.1% | 0.1% |
| Unknown | | | | | |
| Total Related | | NMT 3.0%, | 0.1% | 0.1% | 0.1% |
| Substances | | Area | | | |
| Sodium | | 1.0-2.0 | 1.5 mg/mL | 1.4 mg/mL | 1.4 mg/mL |
| Benzoate | | mg/mL | | | |

### K. Example 11—Stability of Reconstituted Solutions for Drug Product at 1.5 g at 20-25° C.

Table 12 shows the results of 2 week stability studies performed at storage conditions of 20-25° C. for reconstituted DP with 1.5 g crofelemer.

TABLE 12

| | | Test | Time | | |
|---|---|---|---|---|---|
| Quality Attribute | | Specification | 0 weeks | 1 week | 2 weeks |
| Appearance, | | Dark Red | Conforms | Conforms | Conforms |
| Reconstituted | | solution. No | | | |
| Solution | | visible | | | |
| | | insoluble | | | |
| | | matter | | | |
| pH | | 3.5-5.0 | 4.3 | 4.3 | 4.1 |
| Identified | Gallocatechin | | ND | <LOQ | ND |
| Impurities | Epigallocatechin | | <LOQ | ND | 0.1% |
| | Catechin | | ND | ND | ND |
| | Epicatechin | | ND | <LOQ | ND |
| | Procyanidin B1 | | ND | ND | ND |
| | Procyanidin B2 | | ND | ND | ND |
| Individial | | | 0.1% | 0.1% | 0.1% |
| Unknown | | | | | |
| Total Related | | NMT 3.0%, | 0.1% | 0.3% | 0.2% |
| Substances | | Area | | | |
| Sodium | | 1.0-2.0 | 1.5 mg/mL | 1.4 mg/mL | 1.4 mg/mL |
| Benzoate | | mg/mL | | | |

### I. Example 12—Stability of Reconstituted Solutions for Drug Product at 450 mg at 2-8° C.

Table 13 shows the results of 2 week stability studies performed at storage conditions of 2-8° C. for reconstituted DP with 450 mg crofelemer.

TABLE 13

| | | Test | Time | | |
|---|---|---|---|---|---|
| Quality Attribute | | Specification | 0 weeks | 1 week | 2 weeks |
| Appearance, | | Dark Red | Conforms | Conforms | Conforms |
| Reconstituted | | solution. No | | | |
| Solution | | visible | | | |
| | | insoluble | | | |
| | | matter | | | |
| pH | | 3.5-5.0 | 4.4 | 4.3 | 4.3 |
| Identified | Gallocatechin | | ND | ND | ND |
| Impurities | Epigallocatechin | | ND | ND | ND |
| | Catechin | | ND | ND | ND |
| | Epicatechin | | ND | ND | ND |
| | Procyanidin B1 | | ND | ND | ND |
| | Procyanidin B2 | | ND | ND | ND |
| Individial | | | <LOQ | 0.1% | 0.1% |
| Unknown | | | | | |
| Total Related | | NMT 3.0%, | <LOQ | 0.1% | 0.1% |
| Substances | | Area | | | |
| Sodium | | 1.0-2.0 | 1.4 mg/mL | 1.4 mg/mL | 1.4 mg/mL |
| Benzoate | | mg/mL | | | |

### M. Example 13—Stability of Reconstituted Solutions for Drug Product at 450 mg at 20-25° C.

Table 14 shows the results of 2 week stability studies performed at storage conditions of 20-25° C. for reconstituted DP with 450 mg crofelemer.

TABLE 14

| | | Test | Time | | |
|---|---|---|---|---|---|
| Quality Attribute | | Specification | 0 weeks | 1 week | 2 weeks |
| Appearance, | | Dark Red | Conforms | Conforms | Conforms |
| Reconstituted | | solution. No | | | |
| Solution | | visible | | | |
| | | insoluble | | | |
| | | matter | | | |
| pH | | 3.5-5.0 | 4.4 | 4.3 | 4.3 |
| Identified | Gallocatechin | | ND | <LOQ | ND |
| Impurities | Epigallocatechin | | ND | <LOQ | ND |
| | Catechin | | ND | ND | ND |
| | Epicatechin | | ND | ND | ND |
| | Procyanidin B1 | | ND | ND | ND |
| | Procyanidin B2 | | ND | ND | ND |
| Individial | | | <LOQ | 0.1% | 0.1% |
| Unknown | | | | | |
| Total Related | | NMT 3.0%, | <LOQ | 0.1% | 0.1% |
| Substances | | Area | | | |
| Sodium | | 1.0-2.0 | 1.4 mg/mL | 1.4 mg/mL | 1.3 mg/mL |
| Benzoate | | mg/mL | | | |

### N. Example 4—Evaluation of Safety, Tolerability, and Efficacy of Crofelemer Following Multiple Ascending Doses of Drug Product in Participants with Microvillus Inclusion Disease (MVID)

### Study Objectives and Study Endpoints

### Primary Objectives and Endpoints

### Study Objectives

The study will evaluate the safety, tolerability, and preliminary efficacy of multiple ascending doses of a novel crofelemer, compared to placebo, using a randomized cross-over design within each dose level, when administered to pediatric patients with MVID receiving parenteral support (PS, defined as TPN and supplementary IV fluid requirements). Blinded study drug will be administered as a crofelemer formulation, Crofelemer Powder for Oral Solution (i.e. Drug Product), or matching placebo powder for oral solution. Assigned study drug will be reconstituted and administered orally (or enterally) three times daily as a concentrated liquid formulation in each of three dose levels.

### Primary Objectives

To evaluate the safety and tolerability of 3 ascending dose levels of Crofelemer Powder for Oral Solution, compared to placebo, when administered orally or enterally to pediatric participants with MVID receiving PS.

### Primary Endpoints

Assessments of adverse events, serious adverse events, laboratory evaluations, physical exam (including height, weight, BMI and assessment of hydration status), and urine output

### Secondary Objectives and Endpoints

### Secondary Objectives

To evaluate preliminary efficacy of 3 ascending dose levels of Crofelemer Powder for Oral Solution, compared to placebo, when administered orally (or enterally) to pediatric participants with MVID receiving PS with the following specific objectives:
Reduction in loose/watery stool volume and/or frequency and improvement in stool consistency
Improvement in metabolic acidosis
Decreased requirement of weekly total parenteral support (PS; defined as the sum of TPN and IV fluid volume requirements)
Decreased weekly volume requirement of individual PS components namely, a decrease in either TPN or supplemental IV fluid volumes
Decreased requirement for additive electrolytes in IV or TPN fluids

### Secondary Endpoints

Average daily loose/watery stool quantity (by frequency and/or volume) during 24-hour stool collections every two weeks
Average daily loose/watery stool frequency and stool consistency using the 7-point pediatric version of the Bristol Stool Form Scale, m-BSS, every 2 weeks
Stool electrolyte changes (Na⁺, K⁺, Cl⁻) at 20 and 24 weeks
Acetate supplementation in average mEq/mL per week
Average weekly PS volume requirements
Average weekly TPN volume requirements
Average weekly IV fluid supplemental volume requirements
Average weekly supplemental electrolytes in PS and, separately, in TPN and IV fluids

### Exploratory Objectives and Endpoints

### Exploratory Objectives

To evaluate changes in participant/caregiver assessed interpretation of benefit
To evaluate changes in markers of malnutrition and/or caloric requirements over the 20-week crofelemer dose-escalation treatment period

### Exploratory Endpoints

Assessment of change from baseline, compared to placebo, within each dose level over 24 weeks in the following endpoints:
Participant (with help from parent/caregiver) rated PROMIS Scale (v1.0-Gastrointestinal Diarrhea, Napo adapted for MVID)
FACIT-D (Version 4, Napo adapted) participant (with help from caregiver) HRQOL scale
FACIT-G (Version 4, Napo adapted) parent/caregiver HRQOL scale
Plasma albumin, and transferrin levels, and BMI for age will be assessed periodically throughout the study. Additional markers or assessments may be added.
Average weekly calories administered in TPN.

### Investigational Plan

### Overall Study Design

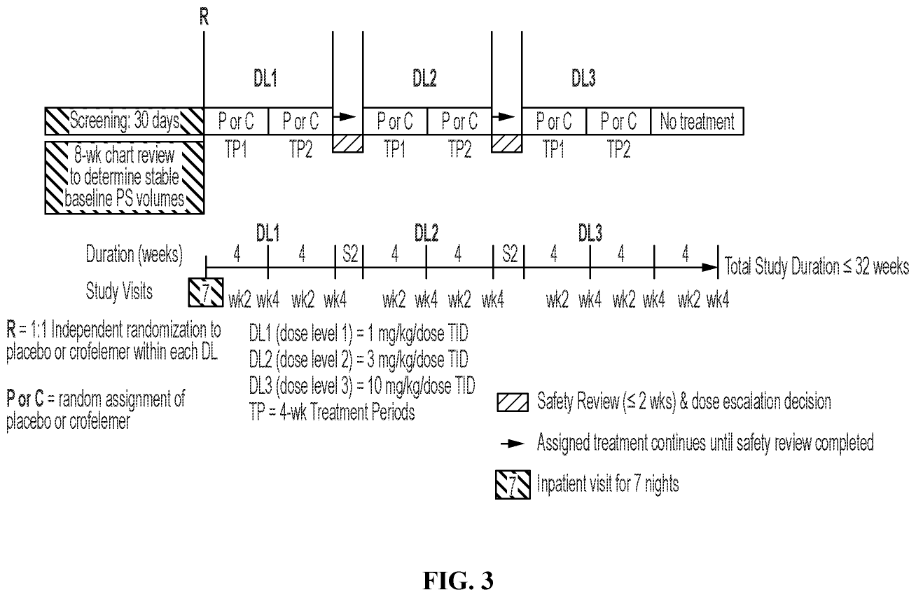

This is a randomized, double-blind, placebo-controlled, dose-escalating study with a placebo cross-over design within each dose level in this ultra-rare MVID participant population (see FIG. 3). For the primary objective, safety and tolerability, comparisons between the crofelemer and placebo within each dose level, and cumulatively over 24 weeks of treatment, will be descriptively summarized. For secondary objectives, changes from the 8-week pre-treatment Baseline Period will be made: 1) within each participant between crofelemer and placebo within each dose level, 2) within each participant between crofelemer and placebo through 24 weeks of treatment, 3) between the crofelemer and placebo groups (if multiple participants per group are enrolled) within each dose level, and 4) between the crofelemer and placebo groups (if multiple participants per group are enrolled) through 24 weeks.

Prior to starting study drug, participants will be randomized to one of 8 randomization sequences which randomly assign 4-week treatment periods (TP) with crofelemer or placebo within each dose level (DL).

TABLE 15

| | | DL1 | | DL2 | | DL3 | | No |
|---|---|---|---|---|---|---|---|---|
| | Sequence | TP1 | TP2 | TP1 | TP2 | TP1 | TP2 | Treatment |
| Randomization | 1 | C | P | C | P | C | P | |
| | 2 | C | P | P | C | C | P | |
| | 3 | C | P | P | C | P | C | |
| | 4 | C | P | C | P | P | C | |
| | 5 | P | C | P | C | P | C | |
| | 6 | P | C | C | P | C | P | |
| | 7 | P | C | C | P | P | C | |
| | 8 | P | C | P | C | C | P | |

DL = dose levels;
TP = treatment periods,
C = crofelemer,
P = placebo

After obtaining informed consent (and assent, if appropriate) and before randomization, study staff will complete an 8-week chart review of the participant's PS requirements to establish the baseline average weekly volume of PS and its components. Average weekly PS volumes (including separate average weekly volumes of TPN and supplemental IV fluid requirements) and average weekly micro- and macronutrients and electrolytes required during the 8-week Baseline Period will be summarized.

Once established that a participant has required stable average weekly PS volumes (no more than a +/−20% difference in ml/kg/week in total PS volume between weeks 1 and 8 of the pretreatment Baseline Period) and meets all eligibility criteria, the participant will be randomized to 1 of 8 possible random sequences of crofelemer or placebo within each of the 3 dose levels. If, during screening and prior to randomization, the potential participant becomes not eligible for the study due to an intercurrent illness (such as gastroenteritis, fever, or line sepsis) and/or instability in average weekly PS volumes, the investigator may wait until the potential participant is stable and meets all eligibility criteria and may collect more recent data to complete all or part of the 8-week pre-treatment Baseline Period.

On study day −2, 3 days before starting study drug (study day 1), the participant will be hospitalized for 7 nights. From Day −2 through Day 0, at least 72 hours of stool and urine will be collected to establish the baseline average daily stool and urine volumes, stool frequency, and stool consistency. During the hospitalization, the participant and parent/caregiver(s) will be instructed on how to collect urine and stool at home for measurement only every 2 weeks, how to complete the PS diary, the stool frequency and consistency diary, and the quality of life diaries.

Each dose level will include 2 4-week treatment periods for a total of 8 weeks. There will be 3 ascending dose levels (DL1, DL2, and DL3) for a total of 24 weeks of treatment with either crofelemer or matching placebo, plus up to 2 additional weeks for safety reviews between dose levels. Within the 24 weeks of treatment, each of crofelemer and placebo will have been administered for at least 12 weeks.

At the end of each 8-week dose level, treatment with the assigned study drug will continue until the Safety Review has been completed to determine if study drug dose may be increased to the next ascending dose level. When a participant advances to the next study dose level, new study drug vials will be dispensed and participant and caregiver(s) will be trained on new study drug dosing instructions as appropriate.

### Review of Safety and Tolerability:

During each of the two 4-week treatment periods within each of the 3 dose levels: Blinded assessment of safety and tolerability since the last visit will be conducted by the investigator and study staff at the 2-week and 4-week visits (including during any optional telehealth visit) within a dose level. (i.e., DL1.TP1.2 wk, DL1.TP1.4 wk, DL1.TP1.2 wk, DL2.TP2.4 wk, DL2.2 wk, DL2.4 wk, DL3.2 wk, and DL3.4 wk).

An internal safety review will be performed to assess safety and tolerability before dose escalation to the next dose level is permitted for each participant.

After escalation to the next dose level: If the higher dose level is not tolerated, the investigator should document the intolerability and may reduce the dose to the previously tolerated lower dose.

### Review of Preliminary Efficacy:

Throughout each 4-week treatment period within each of the three 8-week dose levels, the investigator will assess the efficacy of study drug each week during the usual communications between the parent caregiver with the investigator and study staff as new PS prescriptions are ordered, during study visits, and at the end of each 4-week treatment period. At any time during the 4-week treatment period, the investigator may decide if PS volumes and/or its components should be changed by evaluating one or more of the following that occurred in the previous week and/or throughout the 4-week treatment period: changes in PS volumes (TPN and IV fluids) received by the participant, changes in daily loose/watery stool volume or frequency, stool consistency as measured by m-BSS, urine output (if applicable), and parent and/or investigator assessment of overall hydration status.

Pharmacodynamic assessments will include assessment of changes in the average weekly loose/watery stool volume and average weekly changes in stool consistency (by m-Bristol Stool Form Scale [m-BSS]) at each dose level and cumulatively after 24 weeks of treatment, and changes from baseline in stool electrolytes (Na+, K+, Cl—) through TP1 and TP2 of all 3 dose levels.

Total PS (TPN plus IV fluids) volume requirements, and TPN and IV fluid requirements evaluated separately, at the end of each dosing period and over the entire 24-week treatment period, compared to baseline and to placebo, will be compared.

After completion of dose level 3 (i.e., after the DL3 8-week visit), study drug dosing will be discontinued and the participant will be followed for 4 additional weeks to assess adverse events, changes in stool frequency and/or volume, changes in stool consistency, changes in required PS volumes or PS components, and changes in participant/caregiver assessments of benefit.

An internal safety review considering assessments of clinical observations, safety, tolerability and laboratory evaluations will be performed prior to ascending to the next 8-week study drug dose level.

### Optional Open Label Extension Study

If Crofelemer Powder for Oral Solution is well tolerated and there is evidence of efficacy manifested by a decrease in loose/watery stool frequency and/or volume, or reductions in electrolyte or acetate supplementation requirements, or if IV fluid or TPN volumes can be reduced, participants (and their parent/guardian or caregiver) will be offered the opportunity to participate in a separate open-label extension study under a separate protocol.

### Study Treatment Dose Regimens

Participants will be randomized to 1 of 8 possible random sequences of crofelemer or placebo within each of the 3 dose levels. According to the random sequence order, crofelemer or placebo will be administered for 4 weeks within each dose level. The blinded study drug dose regimens to be administered for each 4-week TP, are as follows:
DL1: crofelemer 1 mg/kg/dose or matching placebo (at same dose volume as crofelemer in DL1) orally TID
DL2: crofelemer 3 mg/kg/dose or matching placebo (at same dose volume as crofelemer in DL2) orally TID
DL3: crofelemer 10 mg/kg/dose or matching placebo (at same dose volume as crofelemer in DL3) orally TID

### Selection and Withdrawal of Subjects

At least two (2) and up to seven (7) pediatric participants with confirmed diagnosis of MVID that require a volume of PS that is 2 50% of their weekly hydration needs for at least the last 8 consecutive weeks prior to baseline will be enrolled.

### Subject Inclusion Criteria

Potential participants are eligible to be included in the study only if all the following criteria apply:
1. Participants (assent for participants older than 7 years of age) and/or their legal parent/guardian sign an Informed Consent Form (ICF) indicating that they understand the purpose of the procedures required for the study and are willing to participate
2. When appropriate, pediatric participants, whose age, cognitive skills, reading abilities and maturity allow the understanding of the study protocol should provide written assent to participate.
3. Male or female participants between the ages of 3 months to 17 years at the time of signing the informed consent or providing assent
4. Have a confirmed diagnosis (genetic and/or histologic) of MVID
5. Are able to ingest reconstituted Crofelemer Powder for Oral Solution either orally (PO) or through a previously-placed G-tube or GJ-Tube (not via J-Tube)
6. Have, during the 8 weeks prior to baseline, a volume of PS that represents at least 50% (2 50%) of the participant's weekly hydration volume requirements
7. If female participants have reached menarche, the participant (and caregiver) agree that the participant will remain abstinent or use two accepted methods of birth control during the course of the treatment period and for an additional 30 days following the last dose of study drug.
8. Male participants (and caregiver) agree that the participant will remain abstinent or use contraception during the course of the treatment period and continue on for an additional 90 days following the last dose of study drug.

### Exclusion Criteria

Potential participants are not eligible to be included in the study if any of the following criteria apply: Within the last 4 weeks before study initiation, participants have
1. had significant changes to PS requirements (i.e., ±>20%)
2. had a new requirement for diuretics
3. had any infection requiring IV antibiotic administration
4. a documented active gastrointestinal infection
5. Initiation of any new anti-diarrheal drug
6. Have previously received an organ transplant
7. Have any currently-diagnosed malignancy
8. Is pregnant or breastfeeding
9. Any investigator determined criteria for inability to participate in this study

### Investigational Product, Dosage, and Mode of Administration

Crofelemer Powder for Oral Solution, 1.5 g (Drug product vials contain 1.5 g of Crofelemer. The drug product is reconstituted with 14 mL of water which results in a final volume of approximately 15 mL and final concentration of 100 mg/mL)
Crofelemer Powder for Oral Solution, 450 mg (Drug product vials contain 450 mg of Crofelemer. The drug product is reconstituted with 14 mL of water which results in a final volume is approximately 15 mL and final concentration of 30 mg/mL)
Placebo Powder for oral solution, 1.5 g (vials contains 1.5 g of placebo. The placebo product is reconstituted with 14 mL of water; which results in a final volume of approximately 15 mL and final concentration of 100 mg/mL)
Placebo Powder for Oral Solution, 450 mg (vials contain 450 mg of placebo. The placebo product is reconstituted with 14 mL of water; which results in a final volume is approximately 15 mL and final concentration of 30 mg/mL)
Excipients of the Drug product (DP) and placebo include sucralose, sodium benzoate, citric acid monohydrate, sodium citrate dihydrate and mannitol (mannitol is used in the 450 mg formulation only). Purified Water is used in the manufacture but is removed during processing.

Blinded study drug will be administered orally (or enterally) three times daily (TID) with or without food for investigational drug and matching placebo for at least 4 weeks each.

Study drug dose levels include:
DL1: 1 mg/kg/dose p.o. or enterally TID or matching placebo
DL2: 3 mg/kg/dose p.o. or enterally TID or matching placebo
DL3: 10 mg/kg/dose p.o. or enterally TID or matching placebo

### O. Example 5—Evaluation of Safety, Tolerability, and Efficacy of Crofelemer Following Multiple Ascending Doses of Drug Product in Participants with Tufted Enteropathy

### Study Objectives and Study Endpoints

### Primary Objectives and Endpoints

### Study Objectives

The study will evaluate the safety, tolerability, and preliminary efficacy of multiple ascending doses of crofelemer, compared to placebo, using a randomized cross-over design within each dose level, when administered to pediatric patients with Tufted Enteropathy receiving parenteral support (PS, defined as TPN and supplementary IV fluid requirements). Blinded study drug will be administered as a novel crofelemer formulation, Crofelemer Powder for Oral Solution, or matching placebo powder for oral solution. Assigned study drug will be reconstituted and administered orally (or enterally) three times daily as a concentrated liquid formulation in each of three dose levels.

### Primary Objectives

To evaluate the safety and tolerability of 3 ascending dose levels of Crofelemer Powder for Oral Solution, compared to placebo, when administered orally or enterally to pediatric participants with Tufted Enteropathy receiving PS.

### Primary Endpoints

Assessments of adverse events, serious adverse events, laboratory evaluations, physical exam (including height, weight, BMI and assessment of hydration status), and urine output

### Secondary Objectives and Endpoints

### Secondary Objectives

To evaluate preliminary efficacy of 3 ascending dose levels of Crofelemer Powder for Oral Solution, compared to placebo, when administered orally (or enterally) to pediatric participants with Tufted Enteropathy receiving PS with the following specific objectives:
Reduction in loose/watery stool volume and/or frequency and improvement in stool consistency
Improvement in metabolic acidosis
Decreased requirement of weekly total parenteral support (PS; defined as the sum of TPN and IV fluid volume requirements)
Decreased weekly volume requirement of TPN
Decreased weekly volume requirement of supplemental IV fluid volumes
Decreased requirement for additive electrolytes in IV or TPN fluids

### Secondary Endpoints

Average daily loose/watery stool quantity (by frequency and/or volume) during 24-hour stool collections every two weeks
Average daily loose/watery stool frequency and stool consistency using the 7-point pediatric version of the Bristol Stool Form Scale, m-BSS, every 2 weeks
Stool electrolyte changes (Na⁺, K⁺, Cl⁻) at 20 and 24 weeks
Acetate supplementation in average mEq/mL per week
Average weekly PS volume requirements
Average weekly TPN volume requirements
Average weekly IV fluid supplemental volume requirements
Average weekly supplemental electrolytes in PS and, separately, in TPN and IV fluids

### Exploratory Objectives and Endpoints

### Exploratory Objectives

To evaluate changes in participant/caregiver assessed interpretation of benefit
To evaluate changes in markers of malnutrition and/or caloric requirements over the 20-week crofelemer dose-escalation treatment period

### Exploratory Endpoints

Assessment of change from baseline, compared to placebo, within each dose level over 24 weeks in the following endpoints:
Participant (with help from parent/caregiver) rated PROMIS Scale (v1.0-Gastrointestinal Diarrhea, Napo adapted for Tufted Enteropathy)
FACIT-D (Version 4, Napo adapted) participant (with help from caregiver) HRQOL scale
FACIT-G (Version 4, Napo adapted) parent/caregiver HRQOL scale
Plasma albumin, and transferrin levels, and BMI for age will be assessed periodically throughout the study. Additional markers or assessments may be added.
Average weekly calories administered in TPN.

### Investigational Plan

### Overall Study Design

This is a randomized, double-blind, placebo-controlled, dose-escalating study with a placebo cross-over design within each dose level in this ultra-rare Tufted Enteropathy participant population (see FIG. 3). For the primary objective, safety and tolerability, comparisons between the crofelemer and placebo within each dose level, and cumulatively over 24 weeks of treatment, will be descriptively summarized. For secondary objectives, changes from the 8-week pre-treatment Baseline Period will be made: 1) within each participant between crofelemer and placebo within each dose level, 2) within each participant between crofelemer and placebo through 24 weeks of treatment, 3) between the crofelemer and placebo groups (if multiple participants per group are enrolled) within each dose level, and 4) between the crofelemer and placebo groups (if multiple participants per group are enrolled) through 24 weeks.

Prior to starting study drug, participants will be randomized to one of 8 randomization sequences which randomly assign 4-week treatment periods (TP) with crofelemer or placebo within each dose level (DL).

TABLE 16

| | | DL1 | | DL2 | | DL3 | | No |
|---|---|---|---|---|---|---|---|---|
| | Sequence | TP1 | TP2 | TP1 | TP2 | TP1 | TP2 | Treatment |
| Randomization | 1 | C | P | C | P | C | P | |
| | 2 | C | P | P | C | C | P | |
| | 3 | C | P | P | C | P | C | |
| | 4 | C | P | C | P | P | C | |
| | 5 | P | C | P | C | P | C | |
| | 6 | P | C | C | P | C | P | |
| | 7 | P | C | C | P | P | C | |
| | 8 | P | C | P | C | C | P | |

DL = dose levels;
TP = treatment periods,
C = crofelemer,
P = placebo

After obtaining informed consent (and assent, if appropriate) and before randomization, study staff will complete an 8-week chart review of the participant's PS requirements to establish the baseline average weekly volume of PS and its components. Average weekly PS volumes (including separate average weekly volumes of TPN and supplemental IV fluid requirements) and average weekly micro- and macronutrients and electrolytes required during the 8-week Baseline Period will be summarized.

Once established that a participant has required stable average weekly PS volumes (no more than a +/−20% difference in ml/kg/week in total PS volume between weeks 1 and 8 of the pretreatment Baseline Period) and meets all eligibility criteria, the participant will be randomized to 1 of 8 possible random sequences of crofelemer or placebo within each of the 3 dose levels. If, during screening and prior to randomization, the potential participant becomes not eligible for the study due to an intercurrent illness (such as gastroenteritis, fever, or line sepsis) and/or instability in average weekly PS volumes, the investigator may wait until the potential participant is stable and meets all eligibility criteria and may collect more recent data to complete all or part of the 8-week pre-treatment Baseline Period.

On study day −2, 3 days before starting study drug (study day 1), the participant will be hospitalized for 7 nights. From Day −2 through Day 0, at least 72 hours of stool and urine will be collected to establish the baseline average daily stool and urine volumes, stool frequency, and stool consistency. During the hospitalization, the participant and parent/caregiver(s) will be instructed on how to collect urine and stool at home for measurement only every 2 weeks, how to complete the PS diary, the stool frequency and consistency diary, and the quality of life diaries.

Each dose level will include 2 4-week treatment periods for a total of 8 weeks. There will be 3 ascending dose levels (DL1, DL2, and DL3) for a total of 24 weeks of treatment with either crofelemer or matching placebo, plus up to 2 additional weeks for safety reviews between dose levels. Within the 24 weeks of treatment, each of crofelemer and placebo will have been administered for at least 12 weeks.

At the end of each 8-week dose level, treatment with the assigned study drug will continue until the Safety Review has been completed to determine if study drug dose may be increased to the next ascending dose level. When a participant advances to the next study dose level, new study drug vials will be dispensed and participant and caregiver(s) will be trained on new study drug dosing instructions as appropriate.

### Review of Safety and Tolerability:

During each of the two 4-week treatment periods within each of the 3 dose levels: Blinded assessment of safety and tolerability since the last visit will be conducted by the investigator and study staff at the 2-week and 4-week visits (including during any optional telehealth visit) within a dose level. (i.e., DL1.TP1.2 wk, DL1.TP1.4 wk, DL1.TP1.2 wk, DL2.TP2.4 wk, DL2.2 wk, DL2.4 wk, DL3.2 wk, and DL3.4 wk).

An internal safety review will be performed to assess safety and tolerability before dose escalation to the next dose level is permitted for each participant.

After escalation to the next dose level: If the higher dose level is not tolerated, the investigator should document the intolerability and may reduce the dose to the previously tolerated lower dose.

### Review of Preliminary Efficacy:

Throughout each 4-week treatment period within each of the three 8-week dose levels, the investigator will assess the efficacy of study drug each week during the usual communications between the parent caregiver with the investigator and study staff as new PS prescriptions are ordered, during study visits, and at the end of each 4-week treatment period. At any time during the 4-week treatment period, the investigator may decide if PS volumes and/or its components should be changed by evaluating one or more of the following that occurred in the previous week and/or throughout the 4-week treatment period: changes in PS volumes (TPN and IV fluids) received by the participant, changes in daily loose/watery stool volume or frequency, stool consistency as measured by m-BSS, urine output (if applicable), and parent and/or investigator assessment of overall hydration status.

Pharmacodynamic assessments will include assessment of changes in the average weekly loose/watery stool volume and average weekly changes in stool consistency (by m-Bristol Stool Form Scale [m-BSS]) at each dose level and cumulatively after 24 weeks of treatment, and changes from baseline in stool electrolytes (Na+, K+, Cl—) through TP1 and TP2 of all 3 dose levels.

Total PS (TPN plus IV fluids) volume requirements, and TPN and IV fluid requirements evaluated separately, at the end of each dosing period and over the entire 24-week treatment period, compared to baseline and to placebo, will be compared.

After completion of dose level 3 (i.e., after the DL3 8-week visit), study drug dosing will be discontinued and the participant will be followed for 4 additional weeks to assess adverse events, changes in stool frequency and/or volume, changes in stool consistency, changes in required PS volumes or PS components, and changes in participant/caregiver assessments of benefit.

An internal safety review considering assessments of clinical observations, safety, tolerability and laboratory evaluations will be performed prior to ascending to the next 8-week study drug dose level.

### Optional Open Label Extension Study

If Crofelemer Powder for Oral Solution is well tolerated and there is evidence of efficacy manifested by a decrease in loose/watery stool frequency and/or volume, or reductions in electrolyte or acetate supplementation requirements, or if IV fluid or TPN volumes can be reduced, participants (and their parent/guardian or caregiver) will be offered the opportunity to participate in a separate open-label extension study under a separate protocol.

### Study Treatment Dose Regimens

Participants will be randomized to 1 of 8 possible random sequences of crofelemer or placebo within each of the 3 dose levels. According to the random sequence order, crofelemer or placebo will be administered for 4 weeks within each dose level. The blinded study drug dose regimens to be administered for each 4-week TP, are as follows:
DL1: crofelemer 1 mg/kg/dose or matching placebo (at same dose volume as crofelemer in DL1) orally TID
DL2: crofelemer 3 mg/kg/dose or matching placebo (at same dose volume as crofelemer in DL2) orally TID
DL3: crofelemer 10 mg/kg/dose or matching placebo (at same dose volume as crofelemer in DL3) orally TID

### Selection and Withdrawal of Subjects

At least two (2) and up to seven (7) pediatric participants with confirmed diagnosis of Tufted Enteropathy that require a volume of PS that is 50% of their weekly hydration needs for at least the last 8 consecutive weeks prior to baseline will be enrolled.

### Subject Inclusion Criteria

Potential participants are eligible to be included in the study only if all the following criteria apply:
1. Participants (assent for participants older than 7 years of age) and/or their legal parent/guardian sign an Informed Consent Form (ICF) indicating that they understand the purpose of the procedures required for the study and are willing to participate
2. When appropriate, pediatric participants, whose age, cognitive skills, reading abilities and maturity allow the understanding of the study protocol should provide written assent to participate.
3. Male or female participants between the ages of 3 months to 17 years at the time of signing the informed consent or providing assent
4. Have a confirmed diagnosis (genetic and/or histologic) of Tufted Enteropathy
5. Are able to ingest reconstituted Crofelemer Powder for Oral Solution either orally (PO) or through a previously-placed G-tube or GJ-Tube (not via J-Tube)
6. Have, during the 8 weeks prior to baseline, a volume of PS that represents at least 50% (2 50%) of the participant's weekly hydration volume requirements
7. If female participants have reached menarche, the participant (and caregiver) agree that the participant will remain abstinent or use two accepted methods of birth control during the course of the treatment period and for an additional 30 days following the last dose of study drug.
8. Male participants (and caregiver) agree that the participant will remain abstinent or use contraception during the course of the treatment period and continue on for an additional 90 days following the last dose of study drug.

### Exclusion Criteria

Potential participants are not eligible to be included in the study if any of the following criteria apply: Within the last 4 weeks before study initiation, participants have
1. had significant changes to PS requirements (i.e., ±>20%)
2. had a new requirement for diuretics
3. had any infection requiring IV antibiotic administration
4. a documented active gastrointestinal infection
5. Initiation of any new anti-diarrheal drug
6. Have previously received an organ transplant
7. Have any currently-diagnosed malignancy
8. Is pregnant or breastfeeding
9. Any investigator determined criteria for inability to participate in this study

### Investigational Product, Dosage, and Mode of Administration

Crofelemer Powder for Oral Solution, 1.5 g (Drug product vials contain 1.5 g of Crofelemer. The drug product is reconstituted with 14 mL of water which results in a final volume of approximately 15 mL and final concentration of 100 mg/mL)
Crofelemer Powder for Oral Solution, 450 mg (Drug product vials contain 450 mg of Crofelemer. The drug product is reconstituted with 14 mL of water which results in a final volume is approximately 15 mL and final concentration of 30 mg/mL)
Placebo Powder for oral solution, 1.5 g (vials contains 1.5 g of placebo. The placebo product is reconstituted with 14 mL of water; which results in a final volume of approximately 15 mL and final concentration of 100 mg/mL)
Placebo Powder for Oral Solution, 450 mg (vials contain 450 mg of placebo. The placebo product is reconstituted with 14 mL of water; which results in a final volume is approximately 15 mL and final concentration of 30 mg/mL)
Excipients of Drug product (DP) and placebo include sucralose, sodium benzoate, citric acid monohydrate, sodium citrate dihydrate and mannitol (mannitol is used in the 450 mg formulation only). Purified Water is used in the manufacture but is removed during processing.

Blinded study drug will be administered orally (or enterally) three times daily (TID) with or without food for investigational drug and matching placebo for at least 4 weeks each.

Study drug dose levels include:
DL1: 1 mg/kg/dose p.o. or enterally TID or matching placebo
DL2: 3 mg/kg/dose p.o. or enterally TID or matching placebo
DL3: 10 mg/kg/dose p.o. or enterally TID or matching placebo

### P. Example 6—Evaluation of Safety, Tolerability, and Efficacy of Crofelemer Following Multiple Ascending Doses of Drug Product in Participants with Short Bowel Syndrome

This study evaluates the efficacy, safety, and tolerability of crofelemer in adult patients with Short Bowel Syndrome (SBS).

### Protocol

Patients to be treated with crofelemer will be randomly assigned to the treatment arm or the control. Patients in the treatment arm will take the Drug Product as described in Example 4 except at doses described below. Patients on the control arm will not receive crofelemer at any time on this study and will receive a placebo powder for oral solution that looks identical to the Drug Product.

### Study Treatment Dose Regimens

Crofelemer or placebo will be administered to subject for 4 weeks within each dose level. The blinded study drug dose regimens to be administered for each 4-week TP, are as follows:
DLL: crofelemer 3 mg/kg/dose or matching placebo (at same dose volume as crofelemer in DL1) orally TID
DL2: crofelemer 10 mg/kg/dose or matching placebo (at same dose volume as crofelemer in DL2) orally TID
DL3: crofelemer 20 mg/kg/dose or matching placebo (at same dose volume as crofelemer in DL3) orally TID

### Selection and Withdrawal of Subjects

### Subject Inclusion Criteria

Potential participants are eligible to be included in the study only if all the following criteria apply:
1. Participants (assent for participants older than 18 years of age)
2. Have a confirmed diagnosis of SBS
3. Require > or =50% of their nutrition from parenteral nutrition
4. Are able to ingest reconstituted Drug Product either orally (PO) or through a previously-placed G-tube or GJ-Tube (not via J-Tube)
5. Female participants agree to remain abstinent or use two accepted methods of birth control during the course of the treatment period and for an additional 30 days following the last dose of study drug.
6. Male participants agree to remain abstinent or use contraception during the course of the treatment period and continue on for an additional 90 days following the last dose of study drug.
7. Be able to maintain their current diet for the duration of the study

### Exclusion Criteria

Potential participants are not eligible to be included in the study if any of the following criteria apply: Within the last 4 weeks before study initiation, participants have
1. had significant changes to PS requirements (i.e., ±>20%)
2. had a new requirement for diuretics
3. had any infection requiring IV antibiotic administration
4. a documented active gastrointestinal infection
5. Initiation of any new anti-diarrheal drug
6. Have previously received an organ transplant
7. Have any currently-diagnosed malignancy
8. Is pregnant or breastfeeding
9. Any investigator determined criteria for inability to participate in this study
10. Have any history of or active neurological, endocrine, cardiovascular, pulmonary, hematological, immunologic, psychiatric, or metabolic disease that is considered clinically significant, is not currently controlled by medication, and is stable as deemed by the Investigator

### Effectiveness Variables:

Primary Outcome Measures: 30% or greater reduction in 24-hour stool volume from baseline or a 10% or greater reduction in PN

All publications, patents, and patent applications cited herein are hereby incorporated herein by reference in their entirety.

### Embodiments

1. A pharmaceutical composition comprising a therapeutically effective amount of crofelemer in a lyophilized powder form.

2. A liquid pharmaceutical composition comprising crofelemer, wherein the liquid composition is obtained by reconstituting a powder formulation comprising crofelemer in lyophilized powder form.

3. The pharmaceutical composition according to embodiment 1 or 2, wherein the pharmaceutical composition further comprises a pharmaceutically acceptable preserving agent, optionally wherein the preserving agent is selected from potassium sorbate, sorbic acid, methyl paraben, ethyl paraben, propyl paraben, and sodium benzoate.

4. The pharmaceutical composition according to embodiment 3, wherein the pharmaceutically acceptable preserving agent is also in a lyophilized powder form.

5. The pharmaceutical composition according to embodiment 3 or 4, wherein the pharmaceutically acceptable preserving agent is sodium benzoate.

6. The pharmaceutical composition according to any one of embodiments 1 to 5, wherein the pharmaceutical composition further comprises a sweetener.

7. The pharmaceutical composition according to embodiment 6, wherein the sweetener is selected from sucralose, sucrose, fructose, glucose, erythritol, martitol, lactitol, sorbitol, mannitol, xylitol, tagatos, trehalose, galactose, ramnorth, ribulose, treose, arabinose, xylose, liquisource, allose, altrose, mannose, idose, lactose, maltose, converted sugar, isotorehalose, neotrehalose, acesulfame potassium, acesulfame acid, aspartame, alitame, saccharin, neohesperidin dihydrocalcone, and cyclamate.

8. The pharmaceutical composition according to embodiment 6 or 7, wherein the sweetener is sucralose.

9. The pharmaceutical composition according to any one of embodiments 6 to 8, wherein the sweetener is in a lyophilized powder form.

10. The pharmaceutical composition according to any one of embodiments 1 to 9, wherein the pharmaceutical composition further comprises one or more buffering agents configured to buffer the solution between pH of about 3.5 and about 5, optionally wherein the buffering agent is an organic acid or optionally wherein the buffering agent is selected from citric acid, citrate, and acetate.

11. The pharmaceutical composition according to embodiment 10, wherein the one or more buffering agents are in a lyophilized powder form.

12. The pharmaceutical composition according to any one of embodiments 1 to 11, wherein the pharmaceutical composition further comprises a bulking agent, optionally wherein the bulking agent is selected from mannitol, glycine, sucrose, raffinose, hydroxyethyl starch (HES), dextran, polyvinylpyrolidone, carboxymethylcellulose, lactose, sorbitol, trehalose, and xylitol.

13. The pharmaceutical composition according to embodiment 12, wherein the bulking agent is in a lyophilized powder form.

14. The pharmaceutical composition according to any one of embodiments 1 to 13, formulated to be administered orally.

15. The pharmaceutical composition according to any one of embodiments 1 to 14, wherein the mass ratio of crofelemer to sodium benzoate is from about 300:1 to about 13:1.

16. The pharmaceutical composition according to any one of embodiments 1 to 15, wherein the mass ratio of crofelemer to sodium benzoate is from about 80:1 to about 13:1.

17. The pharmaceutical composition according to any one of embodiments 1 to 16, wherein the mass ratio of crofelemer to sodium benzoate is from about 70:1 to about 13:1.

18. The pharmaceutical composition according any one of embodiments 7 to 9, wherein the mass ratio of crofelemer to sucralose is from about 10:1 to about 5:1.

19. The pharmaceutical composition according to embodiment 10 or 11, wherein the mass ratio of crofelemer to citric acid is from about 90:1 to about 75:1.

20. The pharmaceutical composition according to embodiments 10 or 11, wherein the mass ratio of crofelemer to citrate is from about 100:1 to about 80:1.

21. The pharmaceutical composition according to embodiments 12 or 13, wherein the mass ratio of crofelemer to mannitol is from about 3:1 to about 1:0.5.

22. The pharmaceutical composition according to any one of embodiments 1 to 21, wherein the pharmaceutical composition comprises about 0 to about 10% moisture content by weight.

23. The pharmaceutical composition according to any one of embodiments 1 to 22, wherein the pharmaceutical composition further comprises sodium benzoate in a mass ratio of crofelemer to sodium benzoate from about 300:1 to about 13:1, sucralose in a mass ratio of crofelemer to sucralose from about 10:1 to about 5:1, citric acid in a mass ratio of crofelemer to citric acid from about 90:1 to about 75:1, citrate in a mass ratio of crofelemer to citrate from about 100:1 to about 80:1, and mannitol in a mass ratio of crofelemer to mannitol from about 3:1 to about 1:0.5, further wherein the sodium benzoate, sucralose, citric acid, citrate, and mannitol are in a lyophilized powder form.

24. The liquid pharmaceutical composition of any one of embodiments 2 to 23, wherein the concentration of crofelemer is from about 2 mg/mL to about 120 mg/mL.

25. The liquid pharmaceutical composition of any one of embodiments 2 to 24, wherein the composition is stable at room temperature for 7, 10, 12, 14, 16, 20, or 30 days.

26. A pharmaceutical composition for use in treating short bowel syndrome (SBS) or diarrhea associated with SBS in a subject comprising administering to a subject in need thereof an amount of the pharmaceutical composition comprising a liquid crofelemer formulation prepared from a dispersible form or powder form of crofelemer.

27. A pharmaceutical composition for use in treating Microvillus Inclusion Disease (MVID in a subject comprising administering to a subject in need thereof an amount of the pharmaceutical composition comprising a liquid crofelemer formulation prepared from a dispersible form or powder form of crofelemer.

28. A pharmaceutical composition for use in treating Tufting Enteropathy or diarrhea associated with Tufting Enteropathy in a subject comprising administering to a subject in need thereof an amount of the pharmaceutical composition comprising a liquid crofelemer formulation prepared from a dispersible form or powder form of crofelemer.

29. The pharmaceutical composition for use according to any one of embodiment 26, 27, or 28 wherein the pharmaceutical composition is according to any one of embodiments 1 to 25.

30. The pharmaceutical composition for use according to any one of embodiment 26 or 29, wherein the SBS is being treated and results from surgical resection of the intestine.

31. The pharmaceutical composition for use according to embodiment 26 or 29, wherein the SBS is being treated and is due to a congenital deficiency.

32. The pharmaceutical composition for use according to any one of embodiments 26 to 31, wherein the subject is a human.

33. The pharmaceutical composition for use according to embodiment 32, wherein the subject is treated for SBS and had an intestinal resection as a result of treatment for Crohn's disease, vascular disease, malignancy, radiation enteritis, trauma, or adhesive obstruction.

34. The pharmaceutical composition for use according to embodiment 31 or 33, wherein the subject has less than 30% of its small bowel remaining, less than 25% of its small bowel remaining, less than 20% of its small bowel remaining, or less than 15% of its small bowel remaining.

35. The pharmaceutical composition for use according to embodiment 34, wherein a portion of the large intestine or colon is also resected.

36. The pharmaceutical composition for use according to any one of embodiments 26 or 29 to 35, wherein the subject does not have all or a portion of the duodenum, the jejunum or the ileum, or a combination thereof.

37. The pharmaceutical composition for use according to any one of embodiments 26 to 36, wherein the pharmaceutical composition is administered to the subject in conjunction with enteral nutrition.

38. The pharmaceutical composition for use according to any one of embodiments 26 to 36, wherein the pharmaceutical composition is administered to the subject in conjunction with parenteral nutrition.

39. The pharmaceutical composition for use according to any one of embodiments 26 to 38, wherein the administration results in improvement in a hepatic and/or biliary disorder associated with the parenteral nutrition and/or results in a reduction in the risk or incidence of liver failure.

40. The pharmaceutical composition for use according to any one of embodiments 26 or 29 to 36, wherein the SBS is treated and the SBS is Type 1 associated with terminal jejunostomy, Type 2 associated with jejunocolic anastomosis, or Type 3 associated with jejunoileotransversostomy SBS.

41. The pharmaceutical composition for use according to embodiment 40, wherein the SBS is Type 1 associated with terminal jejunostomy SBS.

42. The pharmaceutical composition for use according to embodiment 27 or 28, wherein the subject is a newborn (0 to 3 months), an infant of about 3 months to 2 years of age, a toddler from about 2 years to about 6 years of age, a child from about 6 years to about 11 years of age, or an adolescent from about 12 years to about 17 years of age.

43. The pharmaceutical composition for use of any one of embodiments 26 to 42, wherein the subject is administered parenteral nutrition at a first daily amount when beginning administration of the pharmaceutical composition and administration of the pharmaceutical composition reduces the need for administering parenteral nutrition by at least 10%, at least 15%, at least 20%, at least 25%, or at least 30% over the course of about the last 12 weeks of administration of the pharmaceutical composition.

44. The pharmaceutical composition for use according to any one of embodiments 26 to 43, wherein the administered daily dose (mg) of the crofelemer to the subject's weight (kg) is from about 1 mg/kg to about 45 mg/kg per day or about 3 mg/kg to about 36 mg/kg per day or about 6 mg/kg to about 30 mg/kg per day.

45. The pharmaceutical composition for use according to any one of embodiments 26 to 43, wherein the administrated daily dose (mg) of the crofelemer to the subject's weight (kg) is from about 3 mg/kg to about 100 mg/kg per day or about 3 mg/kg to about 30 mg/kg per day.

46. The pharmaceutical composition for use according to any one of embodiments 26 to 45, wherein the pharmaceutical composition is administered 2, 3, 4, 5, or 6 times per day or wherein the pharmaceutical composition is administered with each intake of enteral nutrition.

47. The pharmaceutical composition for use according to any one of embodiments 26 to 46, further comprising decreasing the subject's parenteral nutrition daily amount when the subject's degree of edema has not increased.

48. The pharmaceutical composition for use of embodiment 47, further comprising increasing the subject's enteral nutrition daily amount.

49. The pharmaceutical composition for use according to any one of embodiments 26 to 48, wherein the subject has metabolic acidosis when beginning administration of the pharmaceutical composition and administration of the pharmaceutical composition reduces metabolic acidosis by at least 10%, at least 15%, at least 20%, at least 25%, or at least 30% or eliminates metabolic acidosis over the course of about 12 weeks of administration of the pharmaceutical composition or the last 12 weeks of the administration.

50. The pharmaceutical composition for use according to any one of embodiments 26 to 48, wherein the subject is administered IV fluid volumes at a first daily amount when beginning administration of the pharmaceutical composition and administration of the pharmaceutical composition reduces the daily amount of the IV fluid volume by at least 10%, at least 15%, at least 20%, at least 25%, or at least 30% relative to the first daily amount over the course of about 12 weeks of administration of the pharmaceutical composition or the last 12 weeks of the administration.

51. The pharmaceutical composition for use according to any one of embodiments 26 to 48, wherein the subject is administered parenteral support at a first daily amount when beginning administration of the pharmaceutical composition and administration of the pharmaceutical composition reduces the amount of parenteral support by at least 10%, at least 15%, at least 20%, at least 25%, or at least 30% over the course of about 12 weeks of administration of the pharmaceutical composition or the last 12 weeks of the administration.

52. The pharmaceutical composition for use according to any one of embodiments 26 to 48, wherein a stool electrolyte, Na⁺, K⁺, Cl⁻, or any combination thereof, changes at 16, 18, 20, 22, and 24 weeks post-treatment.

53. The pharmaceutical composition for use according to any one of embodiments 26 to 48, wherein acetate supplementation (mEq/mL) per week is reduced by at least 10%, at least 15%, at least 20%, at least 25%, or at least 30% over the course of about 12 weeks of administration of the pharmaceutical composition or the last 12 weeks of the administration.

What is claimed is:

1. A pharmaceutical composition comprising a therapeutically effective amount of crofelemer in a lyophilized powder form.

2. A liquid pharmaceutical composition comprising crofelemer, wherein the liquid composition is obtained by reconstituting a pharmaceutical composition according to claim 1.

3. The pharmaceutical composition according to claim 1, wherein the pharmaceutical composition further comprises a pharmaceutically acceptable preserving agent, optionally wherein the preserving agent is selected from potassium sorbate, sorbic acid, methyl paraben, ethyl paraben, propyl paraben, and sodium benzoate.

4. The pharmaceutical composition according to claim 3, wherein the pharmaceutically acceptable preserving agent is also in a lyophilized powder form.

5. (canceled)

6. The pharmaceutical composition according to claim 1, wherein the pharmaceutical composition further comprises a sweetener selected from sucralose, sucrose, fructose, glucose, erythritol, martitol, lactitol, sorbitol, mannitol, xylitol, tagatos, trehalose, galactose, ramnorth, ribulose, treose, arabinose, xylose, liquisource, allose, altrose, mannose, idose, lactose, maltose, converted sugar, isotorehalose, neotrehalose, acesulfame potassium, acesulfame acid, aspartame, alitame, saccharin, neohesperidin dihydrocalcone, and cyclamate.

7. (canceled)

8. (canceled)

9. The pharmaceutical composition according to claim 6, wherein the sweetener is in a lyophilized powder form.

10. The pharmaceutical composition according to claim 1, wherein the pharmaceutical composition further comprises one or more buffering agents configured to buffer the solution between pH of about 3.5 and about 5, optionally wherein the buffering agent is an organic acid or optionally wherein the buffering agent is selected from citric acid, citrate, and acetate.

11. (canceled)

12. The pharmaceutical composition according to claim 1, wherein the pharmaceutical composition further comprises a bulking agent, optionally wherein the bulking agent is selected from mannitol, glycine, sucrose, raffinose, hydroxyethyl starch (HES), dextran, polyvinylpyrolidone, carboxymethylcellulose, lactose, sorbitol, trehalose, and xylitol.

13. The pharmaceutical composition according to claim 12, wherein the bulking agent is in a lyophilized powder form.

14. The pharmaceutical composition according to claim 1, formulated to be administered orally.

15. The pharmaceutical composition according to claim 1, wherein the mass ratio of crofelemer to sodium benzoate is from about 300:1 to about 13:1.

16. (canceled)

17. (canceled)

18. The pharmaceutical composition according to claim 6, wherein the mass ratio of crofelemer to sucralose is from about 10:1 to about 5:1.

19. The pharmaceutical composition according to claim 10, wherein the mass ratio of crofelemer to citric acid is from about 90:1 to about 75:1.

20. The pharmaceutical composition according to claim 10, wherein the mass ratio of crofelemer to citrate is from about 100:1 to about 80:1.

21. The pharmaceutical composition according to claim 12, wherein the mass ratio of crofelemer to mannitol is from about 3:1 to about 1:0.5.

22. The pharmaceutical composition according to claim 1, wherein the pharmaceutical composition comprises about 0 to about 10% moisture content by weight.

23. The pharmaceutical composition according to claim 1, wherein the pharmaceutical composition further comprises sodium benzoate in a mass ratio of crofelemer to sodium benzoate from about 300:1 to about 13:1, sucralose in a mass ratio of crofelemer to sucralose from about 10:1 to about 5:1, citric acid in a mass ratio of crofelemer to citric acid from about 90:1 to about 75:1, citrate in a mass ratio of crofelemer to citrate from about 100:1 to about 80:1, and mannitol in a mass ratio of crofelemer to mannitol from about 3:1 to about 1:0.5, further wherein the sodium benzoate, sucralose, citric acid, citrate, and mannitol are in a lyophilized powder form.

24. The liquid pharmaceutical composition of claim 2, wherein the concentration of crofelemer is from about 2 mg/mL to about 120 mg/mL.

25. The liquid pharmaceutical composition of claim 2, wherein the composition is stable at room temperature for 7, 10, 12, 14, 16, 20, or 30 days.

26. A method of treating short bowel syndrome (SBS) or diarrhea associated with SBS in a subject comprising administering to a subject in need thereof an amount of the pharmaceutical composition according to claim 1.

27. A method of treating Microvillus Inclusion Disease (MVID) in a subject comprising administering to a subject in need thereof an amount of the pharmaceutical composition according to claim 1, wherein crofelemer is administered at a dose of about 1 mg/kg to about 45 mg/kg per day or about 3 mg/kg to about 36 mg/kg per day or about 6 mg/kg to about 30 mg/kg per day.

28. A method of treating Tufting Enteropathy or diarrhea associated with Tufting Enteropathy in a subject comprising administering to a subject in need thereof an amount of the pharmaceutical composition according to claim 1, wherein crofelemer at a dose of about 1 mg/kg to about 45 mg/k per day or about 3 mg/kg to about 36 mg/kg per day or about 6 mg/kg to about 30 mg/kg per day.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A method of making a pharmaceutical composition according to claim 1, the method comprising:
(a) providing an aqueous solution comprising crofelemer and pharmaceutically acceptable carrier; and
(b) lyophilizing the solution to form a pharmaceutical composition.

50. A method of making a pharmaceutical composition according to claim 1, the method comprising:
(a) mixing crofelemer, a pharmaceutically acceptable preserving agent selected from potassium sorbate, sorbic acid, methyl paraben, ethyl paraben, propyl paraben, and sodium benzoate, and a sweetener in water, thereby forming a mixture; and
(b) lyophilizing the mixture to form a pharmaceutical composition.

51. The method according to claim 49, wherein lyophilizing the mixture in step b. further comprises:
i. performing a first freezing step on the mixture, thereby forming a first frozen mixture
ii. annealing the first frozen mixture to yield an annealed mixture;
iii. subjecting the annealed mixture to a lowered temperature to maintain the annealed mixture in a frozen state, resulting in a second frozen mixture;
iv. a primary drying step comprising drying the second frozen mixture to yield a first dried mixture;
v. a secondary drying step comprising drying the first dried mixture to yield the pharmaceutical composition; and
vi. sealing a container comprising the pharmaceutical composition.

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. The method according to claim 50, wherein the method comprises:
(a) mixing crofelemer, sodium benzoate in a mass ratio of crofelemer to sodium benzoate from about 300:1 to about 13:1, sucralose in a mass ratio of crofelemer to sucralose from about 10:1 to about 5:1, citric acid in a mass ratio of crofelemer to citric acid from about 90:1 to about 75:1, citrate in a mass ratio of crofelemer to citrate from about 100:1 to about 80:1, and mannitol in a mass ratio of crofelemer to mannitol from about 3:1 to about 1:0.5, thereby forming a mixture;
(b) lyophilizing the mixture to form a pharmaceutical composition, wherein lyophilizing the mixture comprises:
i. performing a first freezing step on the mixture, wherein the first freezing step is performed at a temperature of from about −50° C. to about −30° C. at a temperature ramping rate of about −40° C./hour to about −20° C./hour for a soak time of about 2 to about 4 hours at a chamber pressure of about ≤12 PSIA, thereby forming a first frozen mixture;
ii. annealing the first frozen mixture, wherein annealing is performed at a temperature of from about −10° C. to about −20° C. at a temperature ramping rate of about −20° C./hour to about −40° C./hour for a soak time of about 4 to about 8 hours at a chamber pressure of about ≤12 PSIA, thereby yielding an annealed mixture;
iii. subjecting the annealed mixture to a lowered temperature to maintain the annealed mixture in a frozen state, resulting in a second frozen mixture, wherein subjecting the annealed mixture to a lowered temperature is performed at a temperature of from about −50° C. to about −30° C. at a temperature ramping rate of about −20° C./hour to about −40° C./hour for a soak time of about 2 to about 6 hours at a chamber pressure of about ≤12 PSIA, thereby forming a second frozen mixture;
iv. a primary drying step comprising drying the second frozen mixture, wherein the primary drying step is performed at a temperature of about 20° C. to about 30° C. at a temperature ramping rate of about 20° C./hour to about 40° C./hour for a soak time of about 20 to about 40 hours at a chamber pressure of about 40 microns to about 80 microns, thereby yielding a first dried mixture;
v. a secondary drying step comprising drying the first dried mixture, wherein the secondary drying step is performed at a temperature of about 20° C. to about 30° C. for a soak time of about 10 to about 30 hours at a chamber pressure of about 400 microns to about 800 microns to yield the pharmaceutical composition; and
vi. sealing a container comprising the pharmaceutical composition, wherein sealing a container is performed at about 15° C. to about 35° C. at a chamber pressure of about 5 PSIA to about 10 PSIA.

91. (canceled)

92. (canceled)

93. (canceled)

94. (canceled)

95. (canceled)

96. (canceled)

97. (canceled)

98. (canceled)

99. (canceled)

100. (canceled)
